(19) United States
(12) Patent Application Publication
(10) Pub. No.: US 2024/0392028 A1
(43) Pub. Date: Nov. 28, 2024

(54) METHOD OF TREATING MULTIPLE MYELOMA

(71) Applicant: Caelum Biosciences, Inc., Bordentown, NJ (US)

(72) Inventors: Michael Spector, Bordentown, NJ (US); Susan B. Sobolov-Jaynes, Bordentown, NJ (US)

(21) Appl. No.: 18/691,365

(22) PCT Filed: Sep. 8, 2022
(86) PCT No.: PCT/US2022/042944

§ 371 Date: Mar. 12, 2024

Related U.S. Application Data
(60) Provisional application No. 63/400,882, filed on Aug. 25, 2022.
(60) Provisional application No. 63/285,435, filed on Dec. 2, 2021.
(60) Provisional application No. 63/244,113, filed on Sep. 14, 2021.

(51) Int. Cl. C07K 16/28; A61K 9/00; A61K 39/00; A61K 47/26; A61P 43/00

(52) U.S. Cl. CPC C07K 16/2896; A61K 9/0019; A61K 47/26; A61P 43/00; A61K 2039/507; A61K 2039/545

(57) ABSTRACT

Provided herein are methods of treating multiple myeloma and methods of inhibiting amyloid formation in a subject. The methods provided herein include the administration of an antibody that binds to misfolded light chains in combination with an anti-CD38 antibody. The anti-CD38 is for example Daratumumab, Isatuximab, CID-103 (CASI Pharma), or Moro3087 (Morphosys) or a combination thereof.

### CROSS-REFERENCE TO RELATED APPLICATIONS

This application claims benefit of priority under 35 U.S.C. § 119 (c) of U.S. Provisional Application Nos. 63/244,113, filed Sep. 14, 2021, 63/285,435, filed Dec. 2, 2021, and 63/400,882, filed Aug. 25, 2022. The disclosures of the prior applications are considered part of and are herein incorporated by reference in the disclosure of this application in their entirety.

### FIELD OF THE INVENTION

The present disclosure relates generally to amyloidosis and more specifically to methods of treating multiple myeloma using a combination therapy.

### BACKGROUND

Multiple myeloma is rare cancer of the plasma cells, a type of white blood cell that produces antibodies. Multiple myeloma is a clonal malignancy of terminally differentiated B lymphocytes characterized by the expansion of clonal plasma cells in the bone marrow resulting in suppression of normal hematopoiesis, production of monoclonal immunoglobulins or fragments (light or heavy chain), immunosuppression, nephropathy and neuropathy. Instead of producing normal antibodies, the myeloma cells produce large numbers of a single antibody referred to as monoclonal proteins or M-proteins. This often results in direct injury or accumulation of immunoglobulins (heavy or light chain) in various organs.

Some patients with multiple myeloma also have amyloidosis. The presence of systemic amyloidosis as a co-morbid condition to myeloma and lymphoma and organ involvement is associated with inferior outcomes.

Amyloidosis is a group of diseases that have one thing in common: abnormal protein deposits in bodily tissues, with symptoms that depend on which organs are affected. The most important organs that may be injured by amyloidosis include the heart, kidneys, nerves and liver. One of the most common types of amyloidosis is called AL (immunoglobulin light chain) amyloidosis.

Therapy for AL amyloidosis is similar to that of multiple myeloma, though patients may have a difficult time tolerating therapy due to the symptoms they are experiencing as a result of the underlying amyloidosis. For patients ineligible for stem cell transplantation, targeted chemotherapies to eradicate the underlying plasma-cell dyscrasia (PCD) are used. Such treatments rely on cytotoxic chemotherapy, such as a combination of melphalan and dexamethasone, and a combination of bortezomib and dexamethasone. However, since cytotoxic chemotherapy is, at best, effective only to stop the further production of abnormal antibodies and protein deposits, prognosis remains exceedingly poor due to persistence (or progression) of the pathologic deposits and the absence of improvement and/or reversing of organ dysfunction.

Hence, there is a need for a multiple myeloma therapy that can target myeloma cells, stop the further production of abnormal antibodies, and reduce protein deposits. The compositions and methods disclosed herein fulfill this need.

### SUMMARY

The present disclosure is based on the discovery that the combination therapy of an antibody described herein that binds to misfolded light chains, in combination with an anti-CD38 antibody, for example Daratumumab, Isatuximab, CID-103 (CASI Pharma), or Moro3087 (Morphosys) or a combination thereof, is efficient for the treatment of multiple myeloma. The present disclosure is exemplified but not limited by the disclosure in the Examples herein.

In one embodiment, the disclosure provides a method of treating multiple myeloma in a subject including administering to the subject an antibody having a heavy chain variable domain (VH) as set forth in SEQ ID NO: 1 and a light chain variable domain (VL) as set forth in SEQ ID NO:2; and an anti-CD38 antibody, thereby treating multiple myeloma in the subject. In aspects of this embodiment, the antibody may be administered in a dose from about 500 mg/m² to 1,000 mg/m². In further aspects, a weekly antibody administration dose may be about 500 mg/m² of antibody, about 750 mg/m² of antibody or about 1,000 mg/m² of antibody. The weekly antibody administration dose may include about 10 to 15 mg/kg of antibody, about 15 to 20 mg/kg of antibody or about 20 to 30 mg/kg of antibody. The antibody may be administered weekly for at least 2, 3 or 4 weeks, optionally followed by a maintenance dose of the antibody. The maintenance dose of the antibody may be administered biweekly, triweekly, or monthly after the first 2, 3, 4 or more weeks. In aspects of this embodiment, the anti-CD38 antibody is daratumumab and may be administered in a dose from about 10 to 20 mg/kg, preferably weekly for at least a first or a second cycle. A maintenance dose of the daratumumab to the subject biweekly, triweekly, every four weeks or monthly after the first or second cycle.

The antibody may be administered prior to, simultaneously with, or after administration of the anti-CD38 antibody, e.g., Daratumumab, Isatuximab, CID-103 (CASI Pharma), or Moro3087 (Morphosys) or a combination thereof. Aspects of this embodiment include administration of the antibody and/or the daratumumab by intravenous (IV) infusion, subcutaneous injection or intramuscular injection.

In one embodiment, the disclosure provides a method for inhibiting amyloid formation by administering the antibody disclosed herein to a subject, which in turn binds to misfolded proteins in the circulation, thereby inhibiting amyloid formation.

### BRIEF DESCRIPTION OF THE DRAWINGS

FIGS. 1A-1B show the sequences of the heavy and light chains of the antibody, including the CDR regions. FIG. 1A shows the sequence of the heavy chain. FIG. 1B shows the sequence of the light chain, including the CDR regions.

FIGS. 2A-2C illustrate the characterization of the antibody charge heterogeneity assessed by three independent methods. FIG. 2A shows the antibody charge heterogeneity characterization by capillary zone electrophoresis (CZE) separation. FIG. 2B shows the antibody charge heterogeneity characterization by capillary isoelectric focusing (cIEF) separation. FIG. 2C shows the antibody charge heterogeneity characterization by cation exchange chromatography (CEX).

FIGS. 3A-3B illustrate the comparison of individual antibody concentrations in a phase 2 study as compared to the phase 1b study. FIG. 3A is a line graph showing antibody concentrations in patients. FIG. 3B is a line graph showing antibody concentrations in patients on a semi-log scale.

FIGS. 4A-4B illustrate the comparison of individual antibody mean concentrations in a phase 2 study as compared to the phase 1b study. FIG. 4A is a line graph showing antibody mean concentrations in patients on a linear scale. FIG. 4B is a line graph showing antibody mean concentrations in patients on a semi-log scale.

FIG. 5 is a line graph illustrating the antibody mean concentrations from the Phase 1b study, and the determination of the Cₘᵢₙ.

FIG. 6 is a graph bar illustrating the dose proportionality assessment for Cₘₐₓ/dose.

FIG. 7 is a graph bar illustrating the dose proportionality assessment for Cₘᵢₙ/dose.

FIG. 8 is a graph bar illustrating the dose proportionality assessment for AUCτ/dose.

FIG. 9 illustrates the hematologic response to therapy in the specified subgroups.

FIG. 10 is a graph showing Kaplan-Meier estimates of survival free from major organ Deterioration or Hematologic Progression

FIG. 11 is a graph illustrating overall patient cardiac response over time.

### DETAILED DESCRIPTION

This disclosure relates to the discovery that a combination therapy of the antibodies described herein and daratumumab is effective for the treatment of multiple myeloma.

Before the present compositions and methods are described, it is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

As used in this specification and the appended claims, the singular forms “a,” “an” and “the” include plural references unless the context clearly dictates otherwise. Thus, for example, references to “the method” includes one or more methods, and/or steps of the type described herein, which will become apparent to those persons skilled in the art upon reading this disclosure and so forth.

All publications, patents, and patent applications mentioned in this specification are herein incorporated by reference to the same extent as if each individual publication, patent or patent application were specifically and individually indicated to be incorporated by reference.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, it will be understood that modifications and variations are encompassed within the spirit and scope of the instant disclosure.

In one embodiment, the disclosure provides a method of treating multiple myeloma in a subject including administering to the subject a therapy comprising an antibody that binds to light chain fibrils, in combination with an anti-CD38 antibody such as Daratumumab, Isatuximab, CID-103 (CASI Pharma) or Moro3087 (Morphosys) or a combination thereof, thereby treating multiple myeloma in the subject. In an illustrative example provided herein and, in the specification, Daratumumab is provided as the anti-CD38 antibody.

Multiple myeloma (MM), also known simply as “myeloma” or “plasma cell myeloma,” is a cancer of plasma cells, a type of white blood cell that normally produces antibodies. MM is often initially asymptomatic, with bone pain, anemia, kidney dysfunction, and infections occurring as the disease progresses. There is no known cause for MM, but obesity, radiation exposure, family history and certain chemicals are considered risk factors.

B lymphocytes are produced in the bone marrow and relocate to the lymph nodes upon maturation. As they progress, they mature and display different proteins on their cell surfaces. When they are activated to secrete antibodies, they are known as plasma cells. MM develops in B lymphocytes after they have left the germinal center of lymph node. The normal cell line most closely associated with MM cells is generally taken to be either an activated memory B cell or a precursor to plasma cells, the plasmablast. The immune system keeps the proliferation of B cells and the secretion of antibodies under tight control. Genetic events, such as mutations or translocation can be responsible for important modulation of B cell proliferation that can lead to the development of MM.

MM develops from monoclonal gammopathy of undetermined significance that progresses to smoldering myeloma. Abnormal plasma cells produce abnormal antibodies and/or monoclonal free light chains, which can cause kidney problems and overly thick blood. The plasma cells can also form a mass in the bone marrow or soft tissue. When one tumor is present, it is called a plasmacytoma; the presence of more than one tumor leads to the appellation “multiple myeloma.” MM is diagnosed based on blood or urine tests finding abnormal antibodies, bone marrow biopsy finding cancerous plasma cells, and medical imaging finding bone lesions. Another common finding is high blood calcium levels. Because many organs can be affected by myeloma, the symptoms and signs vary greatly. Fatigue and bone pain are the most common symptoms at presentation. Because of the various effects induced by MM, there are various ways to diagnose the disease. MM can be diagnosed, for example, through blood tests, histopathology, medical imaging or the use of the diagnostic criteria.

Blood test usually rely on the detection of the presence of a paraprotein (monoclonal protein, or M protein and/or monoclonal free light chains); increased levels of all classes of immunoglobulin, especially IgG paraproteins, IgA and IgM; increased levels of isolated light and or heavy chains (κ- or λ-light chains or any of the five types of heavy chains α-, γ-, δ-, ε- or μ-heavy chains); raised calcium level (when osteoclasts are breaking down bone, releasing it into the bloodstream), and/or raised serum creatinine level due to reduced kidney function.

Histopathology can be used to estimate the percentage of bone marrow occupied by plasma cells, by performing a bone marrow biopsy. Characterization of the particular cell types based on the expression of surface proteins can be used to detect plasma cells that express immunoglobulin in the cytoplasm and occasionally on the cell surface. Myeloma cells are often CD56, CD38, CD138 and CD319 positive and CD19, CD20 and CD45 negative. The morphology of the cells can also be studies and used as a distinctive characteristic of myeloma cells.

The diagnostic examination of a person with suspected MM typically includes a skeletal survey or PET-CT. If skeletal survey or PET-CT is negative, a whole-body MRI is performed to detect bone lesions.

Diagnostic criteria have been developed to help the diagnosis of MM. A diagnostic of symptomatic myeloma is asserted when a patient meets at least one of the following criteria: clonal plasma cells account for >10% on a bone marrow biopsy or (in any quantity) in a biopsy from other tissues (plasmacytoma); a monoclonal protein (myeloma protein) is detected in either the serum or urine and it is higher than 3 g/dL (except in cases of true non-secretory myeloma); and evidence of end-organ damage related to the plasma cell disorder (related organ or tissue impairment, CRAB) are found.

The CRAB criteria encompass the most common signs of MM:
Calcium: serum calcium >0.25 mmol/L (>1 mg/dL) higher than the upper limit of normal or >2.75 mmol/L (>11 mg/dL);
Renal insufficiency: creatinine clearance <40 mL per minute or serum creatinine >1.77 mol/L (>2 mg/dL);
Anemia: hemoglobin value of >2 g/dL below the lowest limit of normal, or a hemoglobin value <10 g/dL;
Bone lesions: one or more osteolytic lesion on skeletal radiography, CT, PET/CT or MRI.

For MM, staging helps with prognostication but does not guide treatment decisions. MM can be classified as follows: Stage I: β2 microglobulin (β2M)<3.5 mg/L, albumin ≥3.5 g/dL, normal cytogenetics, no elevated LDH. Stage II: Not classified under Stage I or Stage III. Stage III: β2M≥5.5 mg/L and either elevated LDH or high-risk cytogenetics [t(4,14), t(14,16), and/or del(17p)].

A myeloma protein is an abnormal antibody (immunoglobulin) or (more often) a fragment thereof, such as an immunoglobulin light chain (or a heavy chain), that is produced in excess by an abnormal monoclonal proliferating plasma cell. Other terms for such a protein include M protein, M-component, M-spike, spike protein, monoclonal protein or paraprotein. This proliferation of the myeloma protein has several deleterious effects on the body, including impaired immune function, abnormally high blood viscosity (“thickness” of the blood), and kidney damage.

Myeloma is a malignancy of plasma cells. Plasma cells produce immunoglobulins, each consisting of pairs of heavy and light chains. For MM, a malignant clone, a rogue plasma cell, reproduces in an uncontrolled fashion, resulting in overproduction of the specific antibody the original cell was generated to produce, resulting in a “spike” on the normal distribution, which is called an M spike (or monoclonal spike). Detection of paraproteins in the urine or blood is most often associated with monoclonal gammopathy of undetermined significance (MGUS), a precursor of MM and in MM. An excess in the blood is known as paraproteinemia. Unlike normal immunoglobulin antibodies, paraproteins cannot fight infection.

In one embodiment, the disclosure provides a method of treating MM and/or inhibiting amyloid formation by binding to precursor misfolded proteins in circulation in a subject including administering to the subject an antibody having a heavy chain variable domain (VH) as set forth in SEQ ID NO:1 and a light chain variable domain (VL) as set forth in SEQ ID NO:2; and daratumumab, thereby treating multiple myeloma in the subject. The antibody of this disclosure binds to N-terminus portion of light chain fibrils, precursor misfolded proteins in circulation or amyloid deposits. Without wishing to be bound to theory, the antibody is believed to activate Fc mediated effector functions and facilitate FcR-mediated opsonization through macrophage phagocytosis, thereby destroying myeloma cells. Daratumumab is an IgG1k monoclonal antibody directed against CD38, which is overexpressed in multiple myeloma cells. Daratumumab binds to CD38 on surface of plasma cells and causes apoptosis through several mechanisms including CDC (complement-defendant-cytotoxicity), ADCC (antibody dependent cellular cytotoxicity) and ADCP (antibody dependent cellular phagocytosis) (Phipps, C. et al., Ther. Adv. Hematol., 6:120-7, 2015). Based on the complementary mechanism of the antibody and daratumumab, which both activate macrophages and complement, there is an expected synergistic effect for use in combination to treat MM.

An antibody consists of four polypeptides: two identical copies of a heavy (H) chain polypeptide and two copies of a light (L) chain polypeptide. There are five types of heavy chains: IgG, IgM, IgA, IgD and IgE; and two possible light chains: kappa (κ) and lambda (λ). Each heavy chain contains one N-terminal variable (VH) region and three C-terminal constant (CH1, CH2 and CH3) regions, and each light chain contains one N-terminal variable (VL or V_{K}, or V_{λ} or V_{κ}) region and one C-terminal constant (CL) region. Each variable domain of the light and heavy chain in an antibody also includes three segments called complementarity-determining regions (“CDR”) or hypervariable regions. Each CDR in a light chain, together with the corresponding CDR in the adjacent heavy chain, form an antigen-binding site of the antibody. The variable regions of each pair of light and heavy chains form the antigen binding site of an antibody, whereas the constant region provides structural support and modulates the immune response initiated by the antigen binding.

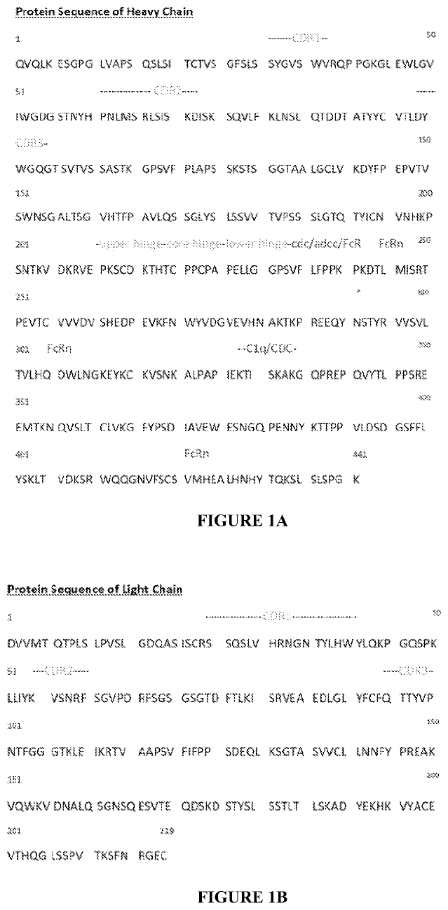

The antibody described herein has a V_{K} region (SEQ ID NO:2) and a V_{H} region (SEQ ID NO:1) as shown in Table 1 below and in FIG. 1A. The CDR sequences for the heavy and light chains and provided in Table 2 and FIG. 1B.

TABLE 1
Monoclonal antibody variable sequences

| | |
|---|---|
| SEQ ID NO: 2 | V_{K} region: |
| | DVVMTQTPLSLPVSLGDQASISCRSSQSLVHRNGN |
| | TYLHWYLQKPGQSPKLLIYKVSNRFSGVPDRFSGS |
| | GSGTDFTLKISRVEAEDLGLYFCFQTTYVPNTFGG |
| | GTKLEIK |
| SEQ ID NO: 1 | V_{H} region: |
| | QVQLKESGPGLVAPSQSLSITCTVSGFSLSSYGVS |
| | WVRQPPGKGLEWLGVIWGDGSTNYHPNLMSRLSIS |
| | KDISKSQVLFKLNSLQTDDTATYYCVTLDYWGQGT |
| | SVTVSS |

TABLE 2
Monoclonal antibody CDR sequences

| | |
|---|---|
| SEQ ID NO: 3 | CDRL1 |
| | SSQSLVHRNGNTYLHWY |
| SEQ ID NO: 4 | CDRL2 |
| | KVSNRF |
| SEQ ID NO: 5 | CDRL3 |
| | QTTYVP |
| SEQ ID NO: 6 | CDRH1 |
| | SYGVSWV |
| SEQ ID NO: 7 | CDRH2 |
| | PNLMSRLSISKD |
| SEQ ID NO: 8 | CDRH3 |
| | DYWGQG |

The genes encoding the V_{H} and V_{K} regions can be cloned to produce a chimeric antibody using known human antibody C_{H} and C_{K} sequences. It is believed that the antibody of the disclosure binds to an epitope expressed by the β-pleated sheet configuration of amyloids, but also to light chain fibrils.

The disclosed antibodies can include any types of human constant regions and/or framework regions. The disclosed humanized and chimeric antibodies, for example, can include the constant regions and/or framework regions of a human IgG (including IgG1, IgG2, IgG3 or IgG4), IgA, IgE, IgF, IgH, or IgM. In one aspect, the disclosed antibody includes a human IgG1 constant region.

Antibodies can be cleaved, for example, with the proteolytic enzyme papain, which causes each of the heavy chains to break, producing three separate antibody fragments. The two identical units that consist of a light chain and a fragment of the heavy chain approximately equal in mass to the light chain are called the Fab fragments (e.g., comprising “antigen binding” fragments). The third unit, consisting of two equal segments of the heavy chain, is called the Fc fragment. The Fc fragment is typically not involved in antigen-antibody binding but is important in later processes involved in elimination of the antigen from the body. “Fv” is the minimum antibody fragment that contains a complete antigen-recognition and—binding site. This region consists of a dimer of one heavy—and one light-chain variable domain in tight, noncovalent association. It is in this configuration that the three CDRs of each variable domain interact to define an antigen-binding site on the surface of the VH-VL dimer. The six CDRs collectively confer antigen-binding specificity to the antibody. Even a single variable domain (or half of an Fv comprising only three CDRs specific for an antigen), however, has the ability to recognize and bind antigen, although at a lower affinity than the entire binding site. “Single-chain Fv” or “sFv” antibody fragments comprise the VH and VL domains of antibody, wherein these domains are present in a single polypeptide chain. The Fv polypeptide can further comprise a polypeptide linker between the VH and VL domains that enables the sFv to form the desired structure for antigen binding. For a review of sFv see Plückthun in The Pharmacology of Monoclonal Antibodies, vol. 113, Rosenburg and Moore eds., Springer-Verlag, New York, pp. 269-315 (1994).

In some aspects, the disclosed antibodies include one or more substitutions, insertions, or deletions, so long as the antibody maintains the ability to bind to amyloid fibrils (e.g., kappa and/or lambda light chain fibrils) or precursor misfolded proteins. The antibody of the present disclosure, for example, can include heavy and light chains with about 85%, about 86%, about 87%, about 88%, about 89%, about 90%, about 91%, about 92%, about 93%, about 94%, about 95%, about 96%, about 97%, about 98%, about 99% or about 100% identity compared to the corresponding heavy and light chain sequences disclosed herein, so long as the antibody maintains the ability to bind to amyloid fibrils or precursor misfolded proteins. In other aspects, the antibody of the present disclosure can include CDRs that have about 85%, about 86%, about 87%, about 88%, about 89%, about 90%, about 91%, about 92%, about 93%, about 94%, about 95%, about 96%, about 97%, about 98%, about 99% or about 100% identity compared to the corresponding CDR sequences disclosed herein, so long as the antibody maintains the ability to bind to amyloid fibrils or precursor misfolded proteins. In some aspects, the antibodies disclosed herein can be engineered to produce differential antigen binding, for example, at varying pH.

The presently disclosed antibodies can bind M proteins or other myeloma paraproteins as well as fibrils and amyloid deposits or precursor misfolded proteins. The binding induces FcR-mediated opsonization of plasma cells through macrophage phagocytosis.

An antibody useful in the compositions and methods of the disclosure may be a monoclonal antibody including CDR sequences of SEQ ID NOs: 3-8. These antibodies bind to an epitope presented by the β-pleated sheet configuration of amyloid fibrils. In one aspect, the antibody of the present disclosure binds to kappa and lambda mis-folded light chains. As used herein “binding to misfolded light chains” refers to the binding specificity of the antibody that recognizes and binds to abnormal light chains (kappa and lambda), but does not recognize and does not bind to non-aggregated or free light chains that are properly folded (in a native and typical conformation) to the same extent. Native light chain or fragments thereof are functional peptides that are normally degraded through proteolysis. Upon mis-folding, a peptide can loss its physiological structure and function; the conformational change renders the peptide non-functional, and more stable, preventing its degradation through proteolysis. Accumulated mis-folded light chains can aggregate with one another to form amyloid fibrils, then can further aggregates with one another or with additional mis-folded light chains. Amyloid fibrils are fibrous deposits that cannot be degraded by the cells and accumulate in plaques around cells to disrupt the healthy function of tissues and organs. Amyloid deposits typically comprise aggregated mis-folded kappa light chain, or mis-folded lambda light chain in a given patient. Such deposits do not usually include both kappa and lambda light chains in an aggregate. The antibody of the present disclosure recognizes both kappa and lambda light chains in their mis-folded conformation and does not recognize kappa or lambda chain in their physiological conformation (properly folded light chains) to the same extent. An aggregate is not required to include both kappa and lambda mis-folded light chains to be recognized by the antibody.

A composition comprising the antibody can include one or more isotonic agents, for example, the composition can include 1, 2, 3 4 or more isotonic agents. In some aspects, the one or more isotonic agents are selected from sugars, poly-alcohols such as mannitol or sorbitol, or sodium chloride. The composition can include a buffer to keep the pH of the composition at a nearly constant value in a wide variety of chemical applications. In some aspects, the buffer is sodium acetate.

The antibody composition can also include a non-ionic surfactant to lower surface tension or interfacial tension. The antibody composition can include, for example, a non-ionic surfactant selected from: ethoxylate, fatty alcohol ethoxylates (such as narrow-range ethoxylate, octacthylene glycol monododecyl ether, and pentaethylene glycol monododecyl ether), alkylphenol ethoxylates (APEs or APEOs, such as nonoxynols and Triton X-100), fatty acid ethoxylates, special ethoxylated fatty esters and oils, ethoxylated amines and/or fatty acid amides (such as polyethoxylated tallow amine, cocamide monoethanolamine, and cocamide diethanolamine), terminally blocked ethoxylates (such as poloxamers), fatty acid esters of polyhydroxy compounds, fatty acid esters of glycerol (such as glycerol monostearate and glycerol monolaurate), fatty acid esters of sorbitol (such as spans: sorbitan monolaurate, sorbitan monostearate, and sorbitan tristearate; and Tweens, or polysorbates: Tween 20, Tween 40, Tween 60, and Tween 80), fatty acid esters of sucrose, and alkyl polyglucosides (such as cecyl glucoside, lauryl glucoside and octyl glucoside).

In one aspect, the antibody composition includes the antibody described herein, sodium acetate, sodium chloride, mannitol and polysorbate 80.

In one aspect, the antibody composition includes from about 20 to 40 mg/ml of antibody. In another aspect, the composition includes from about 15 to 35 mM sodium acetate. In yet another aspect, the composition includes from about 25 to 75 mM sodium chloride. In one aspect, the composition includes from about 0.5 to 5% mannitol. In another aspect, the antibody composition includes from about 0.001 to about 0.1% polysorbate 80. In yet another aspect, the antibody composition has a pH from about 5 to 6.

In one embodiment, the antibody composition includes about 30 mg/mL of antibody; about 25 mM sodium acetate; about 50 mM sodium chloride; about 1% mannitol; about 0.01-0.05% polysorbate 80; and a pH of about 5.5.

In one aspect, the antibody composition includes 30 mg/mL of antibody, about 25 mM sodium acetate, about 50 mM sodium chloride, about 1% mannitol, about 0.01-0.05% polysorbate 80, and has a pH of about 5.5 in a vial or ampule, for example.

In another aspect, the antibody is a mixture of antibody molecules including a native fraction, a reduced fraction, and a glycosylated or deglycosylated fraction having a heterogeneous charge. The mixture of antibody molecules can include those with a native structure (defining a native fraction), a reduced structure (defining a reduced fraction), and a glycosylated or deglycosylated structure (defining a variably glycosylated or deglycosylated fraction), any of which have a heterogeneous charge.

Post-translational modifications (PTMs) induced by chemical and enzymatical intra- and extracellular mechanisms can affect the micro-heterogeneity, and charge heterogeneity of recombinant antibodies and thereby influence important quality attributes, such as stability, solubility, efficacy, safety, pharmacodynamics and pharmacokinetics. The recombinant cell line, the culture media and the process settings may also affect these quality attributes. The distribution of surface charge variants is also an important measure of antibodies' heterogeneity.

Charge variations of proteins differ within the type of modification; some PTMs directly modify the net charge of proteins while others induce conformational changes and variation of local charge distribution. Charge species with a lower isoelectric point (pI) than the main fraction of the product are defined as acidic variants and generated by sialylation, deamidation of asparagine and glutamine, glycation and other mechanisms. Glycation, for instance, is a non-enzymatic reaction where a reducing sugar molecule, most commonly glucose, is covalently bound to a reactive amino group. Basic variants are defined as species with a higher pI than the main fraction and generated by incomplete C-terminal lysine clipping of the heavy chains, as well as by fragmentation and aggregation. Cyclization of N-terminal glutamines to form pyroglutamic acids is another example of positive charge loss of antibodies by the conversion of the N-terminal amine to a neutral amide. Deamidation is a common degradation pathway of proteins that modifies non-enzymatically asparagine residues to aspartic acid and/or isoaspartic residues and/or succinimide intermediates, resulting in the appearance of a negative charge. Some other PTMs affect the local charge distribution without modification of the net charge of the proteins, such as methionine oxidation or aspartic acid isomerization leading to the insertion of an extra methyl group into the backbone protein to form isoaspartic acid. The modifications of charge profiles can potentially affect the structure and the biological activity of proteins. Other PTMs that may impact the functionality of the antibody of the disclosure include fuculose and mannose, which can respectively generate fucosylated and mannosylated antibodies or fragments thereof.

The antibody composition of the present disclosure includes an antibody that can be present in several forms, each form defining a fraction of the antibody composition. The antibody can be present in a native form, for example, which is the main form of the antibody in the absence of any stress, and which represents the main fraction. The antibody can also be present in a reduced form, or in a reduced and deglycosylated form, which represent the reduced fraction and the reduced and deglycosylated fraction, respectively.

In one aspect, the native fraction includes sialylated species, neutral species, and/or galactosylated, fucosylated and/or mannosylated neutral species. Other glycosylated forms might include fucosylated and non-fucosylated forms, and high mannose forms. Since intact antibodies are heterodimeric and contain two heavy chain molecules, the glycosylation on each chain in an intact antibody may be the same or different from that of the other heavy chain. In another aspect, the reduced fraction includes light chains with glycated lysines. As used herein, the phrase “light chain with glycated lysines” is meant to include various levels of glycation of the lysines. No lysine, one lysine, some lysines, or all the lysines of the light chain can be glycated, for example.

Analysis of the surface charge distribution of monoclonal antibodies provides aggregated information about these modifications. Common analytical methods for the determination of charge heterogeneities of antibodies include capillary isoelectric focusing (cIEF) and ion exchange chromatography (IEX). Both methods are widely used, but IEX methods using a salt gradient elution are recognized as the standard and are routinely use. The major limitation of IEX is the salt buffer system, which needs to be adapted for every antibody. The use of pH gradients, however, was shown to be product-independent, and a cation exchange chromatography (CEX) method with a linear pH gradient for the determination of charge heterogeneity of antibody can also be used. These studies report the impact of forced stress degradation at elevated temperature or alkaline pH on mAbs charge variants. Degradations observed with such stresses mainly lead to an increase of acidic species, reflecting deamidation or oxidation reactions of proteins. the charge heterogeneity can also be measured by capillary zone electrophoresis (CZE) separation.

In one aspect, the antibody is a mixture including intact antibodies, halfmer fragments, incomplete antibody fragments, other fragments and/or aggregates thereof. In some aspects, the halfmer is an antibody molecule that includes one or two heavy chains (HC) and one light chain (LC). In other aspects, the incomplete antibody is an antibody missing a C-terminal region of a HC. In some aspects, the other fragment includes HC retaining C-terminal lysine. In various aspects, antibody aggregates, or antibody fragments may or may not retain C-terminal lysine.

“Daratumumab” refers to an antibody that specifically binds CD38, which is overexpressed in MM cells, which is thought to both kill the cancer cells directly and to help the immune system attack them. Daratumumab is marketed under the trade name DARZALEX®. A newer form of the drug is known as daratumumab and hyaluronidase (Darzalex® Faspro®). As used herein, “daratumumab” includes both commercial forms of the drug as well as other formulations containing the daratumumab antibody.

The antibody and daratumumab compositions may be formulated for intravenous, subcutaneous, intraperitoneal, intramuscular, oral, nasal, pulmonary, ocular, vaginal, or rectal administration. In some embodiments, the antibodies and/or daratumumab are formulated for intravenous, subcutaneous, intraperitoneal, or intramuscular administration, such as in a solution, suspension, emulsion, liposome formulation, etc.

Pharmacologically acceptable carriers for various dosage forms are known in the art. Excipients, lubricants, binders, and disintegrants for solid preparations, for example, are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, stabilizing agents and the like.

Additionally, the disclosed pharmaceutical compositions can be formulated as a solution, microemulsion, liposome, or other ordered structure suitable to high drug concentration. The carrier can be, for example, a solvent or dispersion medium containing, for example, water, ethanol, polyol (for example, glycerol, propylene glycol, and liquid polyethylene glycol, and the like), and suitable mixtures thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

In the present disclosure, an antibody of this disclosure and daratumumab are administered to a subject (e.g., a human patient) suffering from MM to promote apoptosis of plasma cells, particularly cancerous myeloma cells. In various embodiments, the present disclosure provides methods of treatment including the administration of the antibody described herein. In some aspects, a therapeutically effective amount of the antibody is administered. A typical route of administration is parenterally (e.g., intravenously, subcutaneously or intramuscularly), as is well understood by those skilled in the medical art. Other routes of administration are, of course, possible. Administration may be by single or multiple doses, alone or in combination with an additional therapy, as discussed below. The amount of antibody administered, and the frequency of dosing may be optimized by the physician for the particular patient.

The methods of the present disclosure can be used for the treatment of MM. As used herein, the phrase “treatment of multiple myeloma” is meant to refer to the reduction of symptoms or indicators of MM in a subject, a decrease in the rate of progression of MM, or improved organ function in MM patients as measured by standard techniques.

Treatment of MM generally relies on the administration of a “therapeutically effective amount” of the therapy described herein, referring to a daratumumab and antibody dose or plasma concentration in a subject, respectively, that provides the specific pharmacological effect for which the therapy is administered in a subject in need of such treatment, e.g., to reduce, ameliorate, or eliminate the symptoms of MM. It is emphasized that a therapeutically effective amount or therapeutic level of a drug will not always be effective in resolving the conditions/diseases described herein, even though such dosage is deemed to be a therapeutically effective amount by those of skill in the art. The therapeutically effective amount may vary based on the route of administration and dosage form, the age and weight of the subject, and/or the subject's condition, including the type and stage of the MM at the time that treatment commences, among other factors.

A therapeutically effective amount may be the dose or amount sufficient to induce a “therapeutic response” in a subject, such as an improvement in at least one measure of MM.

As used herein, the terms “individual,” “patient” or “subject” can be used interchangeably, and refer to an individual organism, a vertebrate, a mammal (e.g., a bovine, a canine, a feline or an equine), or a human that is being administered the therapy of the present disclosure.

The therapy of the present disclosure can be administered to any subject having MM, independently of the treatment previously received, if any, prior to the administration of the presently described therapy. The therapy can be administered whether the MM has been previously treated or has never been treated.

In one aspect, the subject is newly diagnosed with MM prior to administration of the therapy. In other aspects, the subject has been previously treated for MM prior to administration of the therapy.

The terms “administration of” and or “administering” should be understood to mean providing the antibody of the disclosure in a therapeutically effective amount to the subject in need of treatment. Administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. In one aspect, the antibody is administered by intravenous (IV) infusion, subcutaneous injection or intramuscular injection.

The term “cycle” refers to the administration schedule of one or more therapeutics or drugs and refers to the period of time when the one or more therapeutics or drugs is administered to a subject. A cycle may include days in which the drug is administered and periods of rest in which the drug is not administered. Cycle length may vary, and can be for example 1 week, 2 weeks, 3 weeks, 28-days (or 4 weeks), 5 weeks or 6 weeks.

The phrases “combination therapy,” “combined with” and the like refer to the use of more than one medication or treatment simultaneously to increase the response. In combination therapies, daratumumab can be administered prior to, simultaneously with, or following administration of an antibody or antibody composition of the present disclosure. Aspects of this disclosure relate to a combination with additional plasma cell directed therapy such as cyclophosphamide, bortezomib, dexamethasone, melphalan, lenalidomide, isatuximab, venetoclax, a stem cell transplant, or a combination thereof.

Cyclophosphamide is a chemotherapeutic agent that suppress the immune system. Cyclophosphamide can induce the formation of DNA crosslinks both between and within DNA strands at guanine N-7 positions in cells that have low levels of ALDH. DNA crosslinked are irreversible and lead to cell apoptosis. Cyclophosphamide induces beneficial immunomodulatory effects in adaptive immunotherapy, notably by eliminating T regulatory cells (CD4⁺CD25⁺ T cells).

Bortezomib is an anti-cancer medication that binds the catalytic site of the 26S proteasome with high affinity and specificity. By inhibiting the proteasome, bortezomib prevents degradation of pro-apoptotic factors, thereby triggering programmed cell death in neoplastic cells.

Dexamethasone is a corticosteroid medication used in the treatment of many conditions, including rheumatic problems, a number of skin diseases, severe allergies, asthma, chronic obstructive lung disease, croup, brain swelling, eye pain following eye surgery, and along with antibiotics in tuberculosis.

CyBorD is a combination of cyclophosphamide, bortezomib and dexamethasone that is usually used in the treatment of MM.

Melphalan is a chemotherapeutic agent used to treat MM, ovarian cancer, melanoma, and amyloidosis. It is orally or intravenously administered, and chemically alters DNA nucleotide guanine through alkylation. Alkylation causes linkages between strands of DNA that in turn inhibits DNA synthesis and RNA synthesis, and cause cytotoxicity in both dividing and non-dividing tumor cells. Common side effect of melphalan includes bone marrow suppression, which is beneficial for the treatment of amyloidosis.

Lenalidomide is used to treat MM and myelodysplastic syndromes (MDS) and can be administered at least with one other treatment and generally together with dexamethasone.

Isatuximab is a monoclonal antibody used for the treatment of MM. Isatuximab selectively binds to CD38 expressed at the surface of hematopoietic and MM cells, which induces apoptosis of tumor cells and activates immune effector mechanisms such as complement dependent cytotoxicity (CDC), antibody-dependent cellular phagocytosis (ADCP), and antibody-dependent cell-mediated cytotoxicity (ADCC).

Venetoclax is a BH3-mimetic that blocks the anti-apoptotic B-cell lymphoma-2 (Bcl-2) protein, leading to programmed cell death of CLL cells.

As used herein, the phrase “plasma cell directed therapy” is meant to refer to any directed or targeted therapy that can be used to specifically inhibit plasma cells (plasma B cell or antibody-producing cells). Plasma cell-targeted therapies include, but are not limited to lenalidomide, bortezomib, dexamethasone, proteasome inhibitor and combination thereof.

In one aspect, the subject is currently or has been previously treated for MM. In some aspects, a treatment for MM is selected from the group consisting of chemotherapy, corticosteroid, immunomodulating agent, proteasome inhibitor, histone deacetylase (HDCA) inhibitor, immunotherapy, nuclear export inhibitor, stem cell transplant, radiation therapy, surgery, and any combination thereof.

The term “chemotherapy” or “chemotherapeutic agent” as used herein refers to any therapeutic agent used to treat cancer. Chemotherapeutic agent can include any substance or agent having a toxic effect on cells resulting in cell death or reduced proliferation, and especially cancer cell death regardless of the cellular pathway leading to it. Chemotherapy that can be used for the treatment of MM can include, melphalan (a chemotherapeutic agent used to treat MM, ovarian cancer, melanoma, and amyloidosis), vincristine (oncovin), cyclophosphamide (Cytoxan, a chemotherapeutic agent that suppress the immune system), etoposide (vp-16), doxorubicin (adriamycin), liposomal doxorubicin (doxil), or bendamustine (treanda).

Corticosteroids are a class of steroid hormones that are produced in the adrenal cortex of vertebrates, as well as the synthetic analogues of these hormones. Two main classes of corticosteroids, glucocorticoids and mineralocorticoids, are involved in a wide range of physiologic processes, including stress response, immune response, and regulation of inflammation, carbohydrate metabolism, protein catabolismblood electrolyte levels, and behavior. Some common naturally occurring steroid hormones are cortisol, corticosterone, and cortisone. Other examples of corticosteroids include prednisone, prednisolone, dexamethasone, budesonide, beclomethasone dipropionate, triamcinolone acetonide, fluticasone propionate, fluticasone furoate, flunisolide, methylprendisone and hydrocortisone.

Corticosteroids, such as dexamethasone and prednisone are an important part of the treatment of multiple myeloma. They can be used alone or combined with other drugs as a part of treatment. Corticosteroids are also used to help decrease the nausea and vomiting that chemotherapy might cause. Dexamethasone is a corticosteroid medication used in the treatment of many conditions, including rheumatic problems, a number of skin diseases, severe allergies, asthma, chronic obstructive lung disease, croup, brain swelling, eye pain following eye surgery, and along with antibiotics in tuberculosis.

The term “immune modulator” or “immunomodulating agent” as used herein refers to any therapeutic agent that modulates the immune system. Examples of immune modulators include eicosanoids, cytokines, prostaglandins, interleukins, chemokines, checkpoint regulators, TNF superfamily members, TNF receptor superfamily members and interferons. Specific examples of immune modulators include PGI2, PGE2, PGF2, CCL14, CCL19, CCL20, CCL21, CCL25, CCL27, CXCL12, CXCL13, CXCL-8, CCL2, CCL3, CCL4, CCL5, CCL11, CXCL10, IL1, IL2, IL3, IL4, IL5, IL6, IL7, IL8, IL9, IL10, IL11, IL12, IL13, IL15, IL17, IL17, INF-α, INF-β, INF-ε, INF-γ, G-CSF, TNF-α, CTLA, CD20, PD1, PD1L1, PD1L2, ICOS, CD200, CD52, LTα, LTαβ, LIGHT, CD27L, 41BBL, FasL, Ox40L, April, TL1A, CD30L, TRAIL, RANKL, BAFF, TWEAK, CD40L, EDA1, EDA2, APP, NGF, TNFR1, TNFR2, LTBR, HVEM, CD27, 4-1BB, Fas, Ox40, AITR, DR3, CD30, TRAIL-R1, TRAIL-R2, TRAIL-R3, TRAIL-R4, RANK, BAFFR, TACI, BCMA, Fn14, CD40, EDAR XEDAR, DR6, DcR3, NGFR-p75, and Taj. Other examples of immune modulators include tocilizumab (Actemra®), CDP870 (Cimzia®), enteracept (Enbrel®), adalimumab (Humira®), Kineret®, abatacept (Orencia®), infliximab (Remicade®), rituximab (Rituxan®), golimumab (Simponi®), Avonex®, Rebif®, Recigen®, Plegridy®, Betaseron®, Copaxone®, Novatrone®, natalizumab (Tysabri®), fingolimod (Gilenya®), teriflunomide (Aubagio®), BG12, Tecfidera®, and alemtuzumab (Campath®, Lemtrada®).

Immunomodulating agents that can be used to treat multiple myeloma include thalidomide, lenalidomide and pomalidomide.

Thalidomide (Thalomid®) was first used decades ago as a sedative and as a treatment for morning sickness in pregnant women. When it was found to cause birth defects, it was taken off the market but became available again as a treatment for MM. Side effects of thalidomide can include drowsiness, fatigue, severe constipation, and painful nerve damage (neuropathy). The neuropathy can be severe and might not go away after the drug is stopped. There is also an increased risk of serious blood clots that start in the leg and can travel to the lungs.

Lenalidomide (Revlimid®) is similar to thalidomide. It can be used to treat MM. The most common side effects of lenalidomide are thrombocytopenia (low platelets) and low white blood cell counts. It can also cause painful nerve damage. The risk of blood clots is not as high as that seen with thalidomide, but it is still increased. In patients where the myeloma is in remission after either a stem cell transplant or initial treatment, lenalidomide may be given for maintenance therapy to prolong the remission.

Pomalidomide (Pomalyst®) is also related to thalidomide and is used to treat MM. Some common side effects include low red blood cell counts (anemia) and low white blood cell counts. The risk of nerve damage is not as severe as it is with the other immunomodulating drugs, but it is also linked to an increased risk of blood clots.

Proteasome inhibitors work by stopping enzyme complexes (proteasomes) in cells from breaking down proteins important for controlling cell division. They appear to affect tumor cells more than normal cells, but they are not without side effects. Proteasome inhibitor than can be used to treat multiple myeloma include bortezomib, carfilzomib and ixazomib.

Bortezomib (Velcade®) was the first of this type of drug to be approved and is often used to treat MM. It may be especially helpful in treating myeloma patients with kidney problems. In patients where the myeloma was put into remission after either a stem cell transplant or initial treatment, bortezomib may also be given for maintenance therapy to prolong the remission.

Carfilzomib (Kyprolis®) is a newer proteasome inhibitor that can be used to treat MM in patients who have already been treated with other drugs that didn't work. To prevent problems like allergic reactions during the infusion, the steroid drug dexamethasone is often given before each dose in the first cycle.

Ixazomib (Ninlaro®) is a proteasome inhibitor that is a capsule taken by mouth, typically once a week for three weeks, followed by a week off. This drug is usually given after other drugs have been tried.

Histone deacetylase (HDAC) inhibitors are a group of drugs that can affect which genes are active or turned on inside cells. They do this by interacting with proteins in chromosomes called histones. HDAC inhibitor that can be used for the treatment of MM include panobinostat. Panobinostat (Farydak®) is an HDAC inhibitor that can be used to treat patients who have already been treated with bortezomib and an immunomodulating agent. It is a capsule, typically taken three times a week for two weeks, followed by a week off. This cycle is then repeated.

The term “immunotherapy” refers to any type of therapy that includes modulating the immune system or the immune response. Modulating the immune system includes inducing, stimulating or enhancing the immune system as well as reducing, suppressing or inhibiting the immune system. Immunotherapy can be active or passive. Passive immunotherapy relies on the administration of monoclonal antibodies directed against the target to eliminate. Tumor-targeted monoclonal antibodies, for example, have demonstrated clinical efficacy to treat cancer. Active immunotherapy aims to induce a cellular immunity and establish immunological memory against the target agent. Active immunotherapy includes but is not limited to vaccination and immune modulators. Immunotherapy that can be used for the treatment of MM includes monoclonal antibodies such as anti-CD38 antibodies and anti-SLAMF7 antibodies, and antibody-drug conjugates.

Isatuximab (Sarclisa®) is another monoclonal antibody that attaches to the CD38 protein on myeloma cells. This is thought to both kill the cancer cells directly and to help the immune system attack them. This drug is used along with other types of myeloma drugs, typically after at least two other treatments have been tried.

Elotuzumab (Empliciti®) is a monoclonal antibody that attaches to the SLAMF7 protein, which is found on myeloma cells. This is thought to help the immune system attack the cancer cells. This drug is used mainly in patients who have already had other treatments for their myeloma.

The term “antibody-drug conjugate” as used herein refers to a monoclonal antibody linked to a chemotherapy drug. Antibody-drug conjugate for the treatment of MM include an antibody targeting BCMA protein on myeloma cells, and a chemotherapeutic agent. Belantamab mafodotin-blmf (Blenrep®) is an antibody-drug conjugate that can be used by itself to treat myeloma mainly in people who have already had at least four other treatments for their myeloma (including proteasome inhibitors, immunomodulatory drugs, and a monoclonal antibody to CD38).

“Nuclear export inhibitor” or “selective inhibitors of nuclear export” (SINEs) are drugs that block exportin 1 (XPO1 or CRM1), a protein involved in transport from the cell nucleus to the cytoplasm. This inhibition causes cell cycle arrest and cell death by apoptosis, and SINE compounds are of interest as anticancer drugs. Selinexor (Xpovio®) has been approved for treatment of MM as a drug of last resort. It is usually used with dexamethasone.

“Stem cell transplant” or “bone marrow transplant” herein refers to the depletion of a patient of all the cells in his bone marrow (including cancer cells such as myeloma cells) using high-dose chemotherapy, and the transplant of new, healthy blood-forming stem cells. Stem cell transplant is commonly used to treat MM. The transplant can either be autologous, using the patient's own stem cells removed from his or her bone marrow or peripheral blood before the transplant; or allogenic, using blood-forming stem cells from a donor matched to the patient's cell type (such as a close relative to the patient, such as a brother or sister). Stem cell transplant is a standard treatment for patients with MM. Although an autologous transplant can make the myeloma go away for a time (even years), it does not cure the cancer, and the myeloma often returns.

Radiation may be used to treat areas of bone damaged by myeloma that have not responded to chemotherapy and/or other drugs and are causing pain or may be near breaking. It is also the most common treatment for solitary plasmacytomas.

Surgery is sometimes used to remove single plasmacytomas, but it is rarely used to treat MM. When spinal cord compression causes paralysis, severe muscle weakness, or numbness, emergency surgery may be needed. Surgery to attach metal rods or plates can help support weakened bones and may be needed to prevent or treat fractures.

All the additional treatment described herein, that can be used for the treatment of MM can be used alone or in various combination. Among those combinations, the following combinations are often used for the treatment of MM:
Lenalidomide (or pomalidomide or thalidomide) and dexamethasone;
Carfilzomib (or ixazomib or bortezomib), lenalidomide, and dexamethasone;
Bortezomib (or carfilzomib), cyclophosphamide, and dexamethasone;
Elotuzumab (or daratumumab), lenalidomide, and dexamethasone;
Bortezomib, liposomal doxorubicin, and dexamethasone;
Panobinostat, bortezomib, and dexamethasone;
Elotuzumab, bortezomib, and dexamethasone;
Melphalan and prednisone (MP), with or without thalidomide or bortezomib;
Vincristine, doxorubicin (Adriamycin), and dexamethasone (called VAD);
Dexamethasone, cyclophosphamide, etoposide, and cisplatin (called DCEP);
Dexamethasone, thalidomide, cisplatin, doxorubicin, cyclophosphamide, and etoposide (called DT-PACE), with or without bortezomib;
Selinexor, bortezomib, dexamethasone,
Idecabtagene vicleucel, a B-cell maturation antigen-directed chimeric antigen receptor (CAR) T-cell therapy.

The choice and dose of drug therapy depend on many factors, including the stage of the cancer, the age and kidney function of the patient as well as how frail the patient may be. If a stem cell transplant is planned, most doctors avoid using certain drugs, like melphalan, that can damage the bone marrow.

In another embodiment, the disclosure provides a method of treating patients suffering from both MM and another plasma cell disorder including administering to the subject a pharmaceutical composition including an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains and daratumumab.

Plasma cell disorders are a diverse group of disorders of unknown etiology characterized by a disproportionate proliferation of a single clone of B cells and by the presence of a structurally and electrophoretically homogeneous (monoclonal) immunoglobulin or polypeptide subunit in the serum, urine, or both. After developing in the bone marrow, undifferentiated B cells normally enter peripheral lymphoid tissues, such as lymph nodes, spleen, and gut (e.g., Peyer patches) where they begin to differentiate into mature cells, each of which can respond to a limited number of antigens. After encountering the appropriate antigen, some B cells undergo proliferation into plasma cells. Each plasma cell line is committed to synthesizing one specific immunoglobulin antibody that consists of two identical heavy chains (gamma [γ], mu [μ], alpha [α], delta [δ], or epsilon [ε]) and two identical light chains (kappa [κ] or lambda [λ]). A slight excess of light chains is normally produced, and urinary excretion of small amounts of free polyclonal light chains (≤40 mg/24 hours) is normal. Plasma cell disorders are of unknown etiology and are characterized by the disproportionate proliferation of one clone. The result is a corresponding increase in the serum level of its product, the monoclonal immunoglobulin protein (M protein), which can consist of both heavy and light chains or of only one type of chain.

Plasma cell disorders can be classified in two categories: (1) monoclonal gammopathy of undetermined significance, which are usually asymptomatic and associated with monoclonal B or plasma cells, with chronic inflammatory and infection conditions (including chronic cholecystitis, osteomyelitis, pyelonephritis, rheumatoid arthritis and tuberculosis), or associated with other disorders (including familial hypercholesterolemia, Gaucher disease, Kaposi sarcoma, lichen myxedematous, liver disorders, myasthenia gravis, pernicious anemia and hyperthyroidism); and (2) malignant plasma cell disorders, which can either be asymptomatic such as (a) smoldering MM, (b) symptomatic and active MM associated with immunoglobulin and/or light chains production, (c) primary systemic amyloidosis associated with monoclonal light chains (nonhereditary), or associated with heavy chains (IgG, IgA, IgM or IgD heavy chain disease) (d) B-cell lymphoma associated with production of a monoclonal protein.

The most common plasma cell diseases include monoclonal gammopathy of undetermined significance (MGUS, which along smoldering MM is a plasma cell disease in which patients are not yet sick because they have very limited organ damage, if any), MM, and systemic AL amyloidosis. Plasma cell proliferation and M protein production are associated with various symptoms of the diseases including: (1) damage to organs, and particularly the kidneys due to hypercalcemia or toxic light chains secreted by the malignant plasma cell, and due to the fact that some M proteins show antibody activity against self-antigens; (2) impaired immunity, due to the decreased production of other immunoglobulins; (3) bleeding tendency, due to the ability of M protein to coat platelets, inactivate clotting factors, and increase blood viscosity; (4) amyloidosis, due to the ability of M protein and or light chains to form fibrillar deposits within organs (most commonly the heart, kidney and liver); and (5) osteoporosis, hypercalcemia, anemia or pancytopenia, due to the over-activation of osteoclasts by monoclonal plasma cells in bone matrix and/or marrow.

There are many different types of amyloidosis diseases and disorders, including hereditary and sporadic forms, that are caused by outside factors, such as inflammatory diseases or long-term dialysis. In general, amyloidosis is caused by the buildup and aggregation of mis-folded light chain proteins of fragment thereof. Amyloidosis can affect different organs in different people, and there are different types of amyloid. Amyloidosis frequently affects the heart, kidneys, liver, spleen, nervous system and digestive tract. Severe amyloidosis can lead to life-threatening organ failure. Many types affect multiple organs, while others affect only one part of the body. Signs and symptoms of amyloidosis may include, but are not limited to: swelling of the ankles and legs; severe fatigue and weakness; shortness of breath; numbness, tingling or pain in the hands or feet, especially pain in the wrist (carpal tunnel syndrome); diarrhea, possibly with blood, or constipation; unintentional, significant weight loss; an enlarged tongue; skin changes, such as thickening or easy bruising, and purplish patches around the eyes; an irregular heartbeat; or difficulty swallowing.

In an additional embodiment, the disclosure provides a method of identifying a subject having MM as a candidate for an anti-amyloidosis treatment comprising identifying AL amyloidosis fibrils and/or amyloid protein precursor deposition in the subject, wherein the identification of AL amyloidosis fibrils and/or amyloid protein precursor deposition in the subject is indicative of the likelihood of the subject to respond to the therapy, and wherein the therapy includes an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains and daratumumab, thereby identifying the subject as a candidate for the anti-amyloidosis treatment.

As used herein, identifying AL amyloidosis fibrils and/or amyloid protein precursor deposition in the subject can include subjecting the subject to any methods of diagnostic of amyloidosis known in the art. Amyloidosis can be detected in a subject using laboratory tests, biopsies, and/or imaging tests.

Laboratory tests can include blood and urine analysis for the detection of abnormal protein that can indicate amyloidosis. Depending on the signs and symptoms, thyroid and liver function tests may also be indicated. Blood and urine tests can also aid in discovering which organs are involved and how much they are compromised. A 24-hour urine collection to look at the level of protein in a urine sample, for example, can indicate excess protein in the urine, which may be an indication of kidney involvement. Blood test can also be used to test for the presence of abnormal antibody (immunoglobulin) proteins in the blood (to evaluate the level of kappa and lambda light chains).

A tissue biopsy involves the removal of a small sample of tissue to find evidence of amyloid deposits. Any kind of tissue or organ biopsy can be stained with a “Congo-red stain” and analyzed to detect amyloidosis deposits. Less invasive biopsies include fat pad biopsy (from under the skin in the abdomen); labial salivary gland biopsy (the inner lip); and, skin or bone marrow. Bone marrow tests can include bone marrow aspirate (involving the removal of some liquid bone marrow) and bone marrow biopsy (involving the removal of a 1-2 cm core of bone marrow tissue in one piece). These samples can help to determine the percentage of myeloma cells. More invasive biopsy can include organ biopsy, usually performed if amyloidosis is suspected but biopsies of the bone marrow, fat pad, lip or skin sites turn up negative. A surgical biopsy of the organ that is indicating symptoms can then be performed in the liver, kidney, nerve, heart or gut (stomach or intestines).

Imaging test can include echocardiogram and other imaging, that can be used to help establish the extent of the disease. Using an echocardiogram, amyloid deposits can be detected in the heart, while viewing the size and shape of it and the location and extent of any impact of amyloid. Other imaging can include MRI (magnetic resonance imaging), and CMR (for cardiac magnetic resonance), pyrophosphate scanning (a nuclear medicine test also used to evaluate whether an unusual type of cardiomyopathy is present). Nuclear imaging, using radioactive tracers injected to the subject can also be used to reveal early heart damage caused by certain types of amyloidosis. It can also help distinguish between different types of amyloidosis, which can guide treatment decisions. The antibody described herein can also be used for imaging purposes, when coupled to a radioactive tracer such as ¹²⁴I to generate a tagged antibody. Such imaging technique can provide both a localization and extend of deposited amyloid fibrils in the subject. Thus, a labeled antibody of the disclosure may be used to detect the presence of amyloid deposition disease in a patient suspected of having the disease as well as to determine the effectiveness of treatment.

In various aspects, an additional therapy is further administered to the subject.

In some aspects, the additional therapy includes cyclophosphamide, bortezomib, dexamethasone, melphalan, lenalidomide, isatuximab, venetoclax, a stem cell transplant or a combination thereof.

Therapeutically effective doses and dosing regimens of the foregoing methods may vary, as would be readily understood by those of skill in the art. Dosage regimens may be adjusted to provide the optimum desired response (e.g., reduction in the amount of cancerous cells).

In one aspect, the antibody is administered weekly for at least 2, 3 or 4 weeks. Administration of antibody of the disclosure is considered the loading dose, which is the initial dose administered to a subject. The antibody loading dose can for example be followed by a maintenance dose.

In one aspect, a maintenance dose of antibody is further administered to the subject thereafter.

The antibody maintenance dose can be administered at a regimen that is similar to the regimen followed during the loading dose, or the maintenance dose can be administered at a distinct regimen as compared to the regimen followed during the loading dose. The antibody maintenance dose can be administered less often that the loading dose, for example.

In some aspects, the antibody maintenance dose is administered biweekly, triweekly, or monthly after the first 2, 3, 4 or more weeks of weekly administration.

Various other administration regimen may be suitable for the methods described herein. For example, in some aspects, a single dose of the antibody may be administered, while in other aspects, several divided doses may be administered over time, or the dose may be proportionally reduced or increased in subsequent dosing as indicated by the situation. The disclosed antibodies, for example, may be administered once or twice weekly by subcutaneous, intravenous, or intramuscular injection. In some aspects, the disclosed antibodies may be administered once or twice monthly by subcutaneous, intravenous, or intramuscular injection. In some aspects, the disclosed antibodies may be administered once or twice annually by subcutaneous, intravenous, or intramuscular injection. In other aspects, the disclosed antibodies or antigen-binding fragments thereof may be administered once every week, once every other week, once every three weeks, once every four weeks, once every month, once every other month, once every three months, once every four months, once every five months, once every six months, once every seven months, once every eight months, once every nine months, once every ten months, once every eleven months, twice a year, or once a year, as the situation or condition of the patient may indicate.

In one aspect, daratumumab is administered weekly for at least a first cycle or second cycle. A cycle may include days in which the drug is administered and periods of rest in which the drug is not administered. Cycle length may vary, and can be for example 1 week, 2 weeks, 3 weeks, 28-days (or 4 weeks), 5 weeks or 6 weeks

Administration of daratumumab of the disclosure is considered the loading dose, which is the initial dose administered to a subject. The loading dose can for example be followed by a maintenance dose.

The daratumumab maintenance dose can be administered at a regimen that is similar to the regimen followed during the loading dose, or the maintenance dose can be administered at a distinct regimen as compared to the regimen followed during the loading dose. The maintenance dose can be administered less often that the loading dose, for example.

In some aspects, the daratumumab maintenance dose is administered biweekly, triweekly, every four weeks or monthly after the first cycle or second cycle.

Various other administration regimen may be suitable for the methods described herein. A single dose of daratumumab, for example, may be administered, while in other aspects, several divided doses may be administered over time, or the dose may be proportionally reduced or increased in subsequent dosing as indicated by the situation. The first daratumumab administration, for example, may be administered as divided doses over two days by subcutaneous, intravenous, or intramuscular injection. In some aspects, the daratumumab may be administered once or twice monthly by subcutaneous, intravenous, or intramuscular injection. In some aspects, the daratumumab may be administered once or twice annually by subcutaneous, intravenous, or intramuscular injection. In other aspects, the daratumumab may be administered once every week, once every other week, once every three weeks, once every four weeks, once every month, once every other month, once every three months, once every four months, once every five months, once every six months, once every seven months, once every eight months, once every nine months, once every ten months, once every eleven months, twice a year, or once a year, as the situation or condition of the patient may indicate.

The therapeutically effective dose of the combination therapy administered to the patient (whether administered in a single dose or multiple doses) should be sufficient to reduce the amount of cancer cells in the patient. Such therapeutically effective amount may be determined by evaluating the symptomatic changes in the patient or by evaluating the change in the amount of myeloma cells, plasmacytoma size and number, or abundance of paraproteins or M proteins.

An effective dosage for a particular subject may be influenced by attributes such as gender, body surface area (BSA), weight and the like. For a male who is 5′8″ and 150 lbs and having a BSA 1.8 (Mosteller), for example, an effective dose of the antibody may be around 1800 mg. For a male who is 5′ 8″ and 235 lbs and having a BSA 2.3 (Mosteller), for example, an effective dose of the antibody may be about 2300 mg.

Exemplary doses can vary according to the size and health of the individual being treated, as well as the condition being treated. In some aspects, a therapeutically effective amount of a disclosed antibody may be from about 250 mg/m² to 1375 mg/m² or about 500 mg/m² to 1000 mg/m²; however, in some situations the dose may be higher. For instance, in some embodiments, the therapeutically effective amount may be about 1000, about 975, about 950, about 925, about 900, about 875, about 850, about 825, about 800, about 775, about 750, about 725, about 700, about 675, about 650, about 625, about 600, about 575, about 550, about 525, or about 500 mg/m².

In one aspect, an antibody administration dose is from about 500 mg/m² to 1,000 mg/m². In many aspects, the antibody administration dose is selected from about 500 mg/m², about 750 mg/m² and about 1,000 mg/m².

Similarly, in some aspects, the effective amount of an antibody is about 2,200 mg; however, in some situations the dose may be higher or lower. In some embodiments, a therapeutically effective amount may be between about 50 and 5000 mg, between about 60 about 4500 mg, between about 70 and 4000 mg, between about 80 and 3500 mg, between about 90 and 3000 mg, between about 100 and 2500 mg, between about 150 and 2000 mg, between about 200 and 1500 mg, between about 250 and 1000 mg, about 1400 mg and 2300 mg or any dose in between. For instance, in some embodiments, the therapeutically effective amount may be about 50 about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1420, about 1500, about 1600, about 1700, about 1800, about 1900, about 2000, about 2100, about 2200, about 2270, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000 or more mg.

In one aspect, administering a weekly dose of about 1,000 mg/m² of an antibody includes administering about 2,750 mg of the antibody.

In other aspects, administering a weekly dose of about 500 mg/m² of an antibody includes administering about 1,375 mg of the antibody, 510 mg/m² includes administering about 1400 mg of the antibody, administering a weekly dose of about 750 mg/m² of an antibody includes administering about 2,065 mg of the antibody and about 836 mg/m² of antibody includes administering about 2300 mg antibody. In other aspects, an administration dose of about 500 mg/m² of antibody comprises administering about 1,000 to 1,500 mg of antibody, administering about 750 mg/m² of antibody comprises administering about 1,500 to 2,500 mg of antibody, and administering about 1,000 mg/m² of antibody comprises administering about 2,500 to 3,000 mg of antibody. While not wanting to be held to a specific theory, it is believed that approximately 2.75× the mg/m² dosage would be acceptable.

Similarly, in some aspects, the effective amount of an antibody is about 25 mg/kg or about 18.75 mg/kg; however, in some embodiments, the concentration may be higher or lower. In some embodiments, the effective amount may be about 1-50 mg/kg, about 5-40 mg/kg, about 6 to 32 mg/kg, about 10-30 mg/kg, or about 15-25 mg/kg or any value in between. For instance, in some embodiments, the effective amount may be 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, 41, 42, 43, 44, 45, 46, 47, 48, 49, or 50 or more mg/kg.

In one aspect, administering about 1,000 mg/m² of an antibody includes administering about 25 mg/kg of the antibody. In other aspects, an administration dose of about 500 mg/m² of an antibody includes about 12.5 mg/kg of the antibody, and an administration dose of about 750 mg/m² includes about 18.75 mg/kg of the antibody. In other aspects, an administration dose of about 500 mg/m² of antibody comprises about 10 to 15 mg/kg of antibody, a weekly dose of about 750 mg/m² comprises about 15 to 20 mg/kg of antibody and a weekly dose of about 1,000 mg/m² of antibody comprises about 20 to 30 mg/kg of antibody.

In one aspect, an administration dose of daratumumab is from about 10 to 20 mg/kg, about 14 to 18 mg/kg, or any value in between. An effective dose may be about 16 mg/kg.

In other aspects, daratumumab may be administered subcutaneously in a formulation of 1500 mg to 2200 mg of daratumumab per 15 mL, 1600 mg to 2000 mg of daratumumab per 15 mL; 1700 mg to 1900 mg of daratumumab per 15 mL. An effective amount may be about 1800 daratumumab per 15 mL.

The disclosed methods of treatment may also be combined with other known methods of treatment as the situation may require. Thus, in some aspects, the disclosed therapy may be administered prior to, after, or concurrently with other known treatments. In some aspects, the disclosed antibodies may be administered only after other treatment options have failed or the disease has continued to progress.

As discussed above, the disclosed therapy can be administered in combination with an additional therapy. In one aspect, the antibody is administered prior to, simultaneously with, or after the additional therapy.

In various aspects, the disclosed therapy is administered before the additional therapy.

The methods described herein rely on the administration of the antibody of the present disclosure and daratumumab. In various aspects, administering the antibody to a subject includes administering to the subject a pharmaceutical composition including the antibody, sodium acetate, sodium chloride, mannitol, and polysorbate 80.

Beside its efficacy to treat a disease of interest, a therapeutic medication can be associated with other events not related to the treatment of such disease, which can include side effects, toxicities, or adverse events. Therapeutic medication(s), for example, can be associated with dose-limiting toxicity, when an increase of the dose is associated with an increase in the observed toxicity, which can limit or prohibit the use of therapeutically effective doses.

Some therapy may be associated with treatment-emergent adverse events (TEAEs), that were not present prior to the initiation of the treatment or that worsened in either intensity or frequency following exposure to the treatment. Common TEAEs include but are not limited to nausea, diarrhea, urinary trac infection, pain, dizziness, headache, fatigue and insomnia. As used herein, the term “serious adverse event” means an untoward medical event that results in death, is life-threatening, requires inpatient hospitalization or prolongation of existing hospitalization, or results in persistent or significant disfigurement or disability.

In one aspect, the 500 mg/m², 750 mg/m² and 1,000 mg/m² administration doses of antibody and 10 to 20 mg/kg and 14 to 18 mg/kg administration dose of daratumumab do not induce drug-related adverse events.

In another aspect, the 500 mg/m², 750 mg/m² and 1,000 mg/m² doses of antibody and 10 to 20 mg/kg and 14 to 18 mg/kg administration dose of daratumumab do not induce dose limiting toxicities.

The efficacy of the therapy to treat amyloidosis can also be measured based on pharmacokinetics parameters of the therapy.

The efficacy of an antibody dose, for example, can be measured as its ability to bind its target, such as aggregates of A-light chain fibrils and/or K-light chain fibrils. The efficacy of a daratumumab dose can be measured as its ability to bind its target, such as CD38.

The efficacy of a therapy dose can be measured as the site occupancy of the target receptor. The site occupancy of a target receptor can indicate for a given dose of the antibody and/or daratumumab, which proportion of the light chain fibrils or plasma cells are bound to the antibody or daratumumab, and therefore actively target for degradation and apoptosis.

The therapy doses herein can for example be sufficient to induce at least 50% occupancy of the target. The antibody can induce at least 50%, 60%, 70%, 80%, 90%, 95%, 96%, 97%, 98%, 99% or more occupancy of the amyloid deposits or light chain fibrils in a subject.

In one aspect, the 500 mg/m², 750 mg/m² and 1,000 mg/m² administration doses of antibody achieve a site occupancy of a target receptor of at least 90%.

The efficacy of a therapy dose can be measured as its concentration as measured in the subject, as compared to the administration dose.

In one aspect, the concentration of a therapy in the subject increases with the administration dose.

The efficacy of an antibody dose can be measured as the ability to efficiently destroy MM cells a subject.

The following examples are given to illustrate the present disclosure. It should be understood, however, that the disclosure is not to be limited to the specific conditions or details described in these examples. All printed publications referenced herein are specifically incorporated by reference.

Presented below are examples discussing the efficacy of high doses of the antibody of the present disclosure, alone or in combination with a plasma cell directed therapy, contemplated for the discussed applications. The following examples are provided to further illustrate the embodiments of the present disclosure but are not intended to limit the scope of the disclosure. While they are typical of those that might be used, other procedures, methodologies, or techniques known to those skilled in the art may alternatively be used.

### EXAMPLES

### Example 1

### Antibody Production and Characterization

The antibody of the disclosure was produced by transfecting a host cell with a plasmid encoding a codon-optimized DNA sequence to improve translation efficiency a improve transcription efficiency, without altering the amino acid sequence of the antibody.

Cells were cultured in conditions to reach high antibody titer and cell density. The manufacturing process included production in a bioreactor until the optimal balance of cell debris/harvestability and antibody titer was reached.

The antibodies obtained were then characterized by studying charge heterogeneity under various stress conditions.

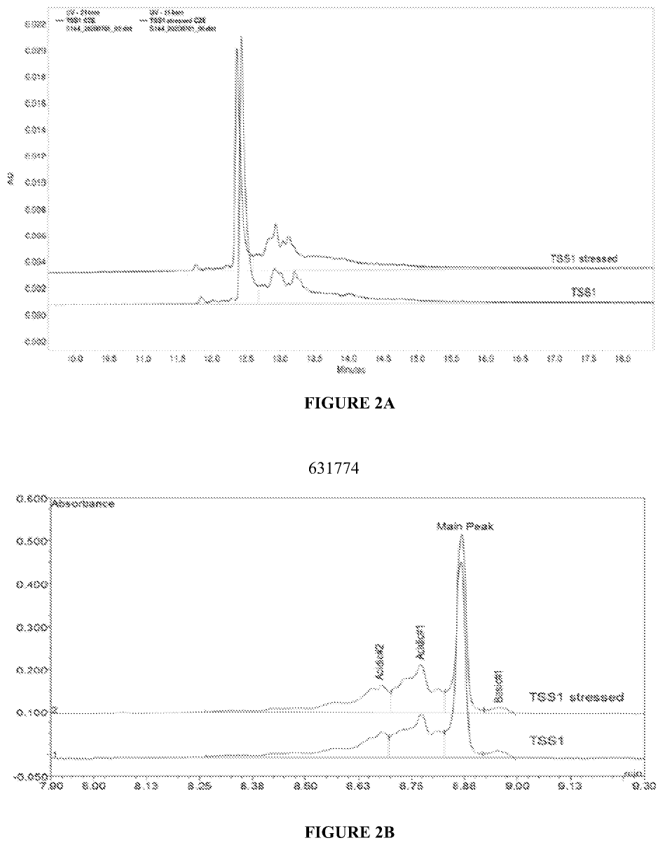

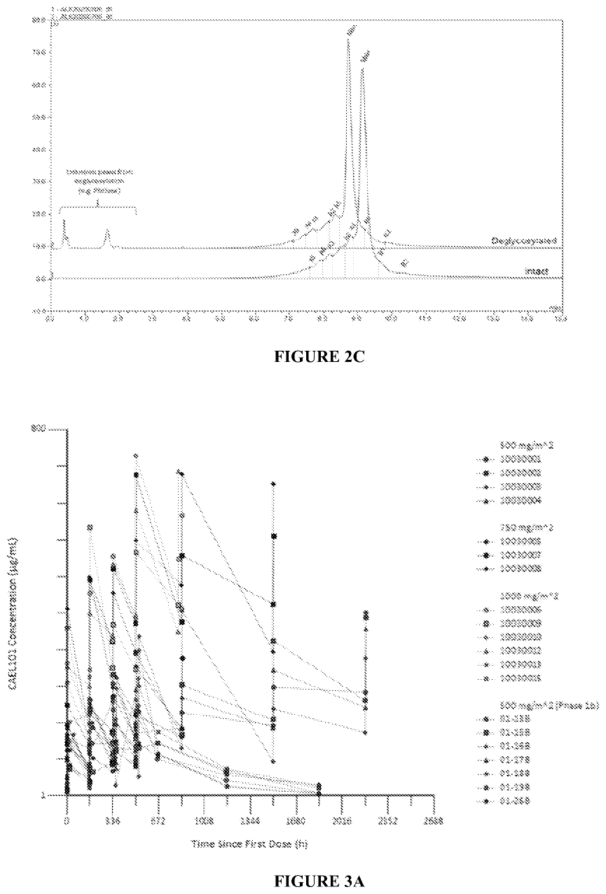

As illustrated in FIG. 2, the analysis of native, reduced, and reduced+deglycosylated fractions showed that all fractions were complex mixtures. All native fractions contained mixtures of the expected glycosylation variants.

AV4 & AV5 fractions contained the sialylated species. The main peak and BV1 fractions were enriched for the smaller neutral species (GO & GOF). It was also shown that more acid fractions were enriched for galactosylated neutral species (GIF & G2F), and that the native AV5 fraction was enriched for halfmers (HC/LC), antibody missing N-terminal half of one HC, and other unidentified fragments. The native BV1 fraction was enriched for HC retaining C-terminal lysine, as expected; and the reduced AV3-5 LC fractions were enriched for glycated lysines.

As illustrated in FIG. 2, the antibody charge heterogencity was assessed by capillary zone electrophoresis (CZE) separation (FIG. 2A), by capillary isoelectric focusing (cIEF) separation (FIG. 2B) and by cation exchange chromatography (CEX, FIG. 2C). The results shown that the apparent heterogencity was not an artifact of a given method.

### Example 2

### Preliminary Results of the Antibody Phase 2 Pharmacokinetic Analysis

The objectives of the preliminary phase 2 pharmacokinetic (PK) data analysis were (1) to assess dose-proportionality in PK exposures from 500 to 1000 mg/m²; (2) to assess the lowest dose/smallest dose number to reach a target C_{trough} of 130 μg/mL; and (3) to assess Phase 3 dose and regimen recommendation with partial Phase 2 PK data.

TABLE 3
Comparison of the Phase 2 vs. Phase 1b study

| | Phase 1b | Phase 2 |
|---|---|---|
| Patient | Patients with AL | Similar |
| | amyloidosis who were | |
| | previously treated | |
| Antibody | Not optimal | New higher producing cell line |
| Material | | New manufacturer with platform |
| | | process for scalability |
| | | Removal of any animal components |
| | | Comparable with Phase 1 material |
| | | by analytical and animal exposure |
| | | (PK) assessments |
| CyBorD | Some had pre-dose | Added CyBorD: chemotherapy to |
| | chemo | be used in Phase 3 dosing |
| PK Assay | Non-validated research | |
| | level ELISA at KCAS: | |
| | LLOQ = 0.5 μg/ml | |

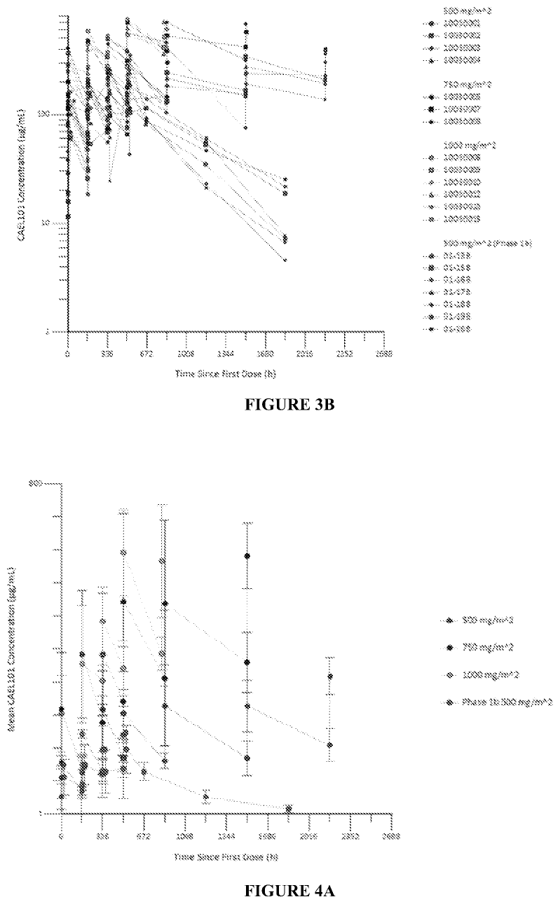

Individual PK of the antibody of the present disclosure was evaluated over time and compared to the data of the Phase 1b study. As illustrated in FIGS. 3A and 3B, in the phase 2 study, 500, 750, and 1000 mg/m² doses had complete peak (highest concentration of the antibody in the patient's bloodstream) and trough (lowest concentration in the patient's bloodstream before administration) data for dose 6, 4, and 3, respectively), and shown an increase in PK exposure from 500 to 1000 mg/m². This is in contrast with the data from the phase 1b study, where concentrations were lower and variability higher.

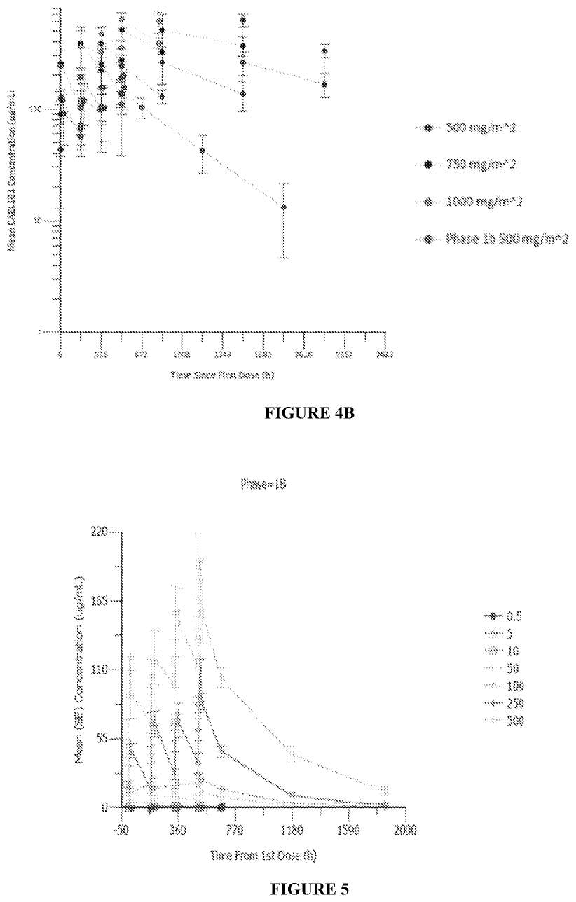

As further shown in FIGS. 4A and 4B study of mean PK of the antibody was evaluated over time and compared to the data of the Phase 1b study. In the phase 2,500,750, and 1000 mg/m² doses had complete peak and trough data for dose 6, 4, and 3, respectively and shown an increase in PK exposure with dose from 500 to 1000 mg/m². This is in contrast with the data from the phase 1b study, where concentrations were lower and variability higher, especially after doses 2 to 4.

The study of the antibody peak exposure Cₘₐₓ (μg/mL) by dose was further evaluated. As shown in Table 4, at 500 mg/m² overall the peak exposures were comparable between Phase 2 and Phase 1b studies. There were overlapping Cₘₐₓ after doses 1 and 4, and similar dose 3/dose 1 accumulation ratio of ˜1.43 to 2-fold. The phase 2 had approximately 50% higher Cₘₐₓ after doses 2 and 3 as compared to the Phase 1b.

In this phase 2 study, there was low to moderate variability. At 1000 mg/m², the last 3 data point added variability (lower PK; slightly more severe disease status). At 750 mg/m², the highest % CV was due to data point 1003-0005, a subject with Cardiac, Mayo Stage 2, who was switched to daratumumab.

TABLE 4

| | Dose 1 | | | Dose 2 | | | Dose 3 | | |
|---|---|---|---|---|---|---|---|---|---|
| | 500 | 750 | 1000 | 500 | 750 | 1000 | 500 | 750 | 1000 |
| Cₘₐₓ | mg | mg | mg | mg | mg | mg | mg | mg | mg |
| N | 4 | 3 | 6 | 4 | 3 | 6 | 4 | 3 | 3 |
| Mean | 126 | 255 | 244 | 194 | 387 | 363 | 253 | 387 | 468 |
| SD | 16.7 | 136.5 | 92.8 | 9.8 | 153.3 | 140.5 | 14.3 | 147.0 | 81.7 |
| Min | 113 | 154 | 134 | 182 | 210 | 210 | 236 | 220 | 374 |
| Median | 120 | 200 | 267 | 196 | 472 | 339 | 254 | 444 | 505 |
| Max | 150 | 410 | 367 | 204 | 479 | 586 | 267 | 497 | 524 |
| CV % | 13% | 54% | 38% | 5% | 40% | 39% | 6% | 38% | 17% |
| GeoMean | 125.0 | 232.9 | 227.1 | 194.1 | 362.1 | 341.5 | 252.4 | 364.8 | 462.6 |
| GeoCV % | 12.7% | 54.1% | 44.4% | 5.1% | 49.9% | 39.8% | 5.7% | 46.4% | 18.7% |

| | Dose 4 | | | Dose 5 | | | Dose 6 | | |
|---|---|---|---|---|---|---|---|---|---|
| | 500 | 750 | 1000 | 500 | 750 | 1000 | 500 | 750 | 1000 |
| Cₘₐₓ | mg | mg | mg | mg | mg | mg | mg | mg | mg |
| N | 4 | 3 | 3 | 4 | 3 | 3 | 4 | 2 | 0 |
| Mean | 244 | 514 | 633 | 263 | 510 | 613 | 262 | | |
| SD | 102.3 | 212.1 | 105.3 | 98.1 | 200.5 | 96.0 | 62.5 | | |
| Min | 138 | 283 | 533 | 184 | 302 | 517 | 192 | | |
| Median | 245 | 559 | 624 | 231 | 526 | 613 | 258 | | |
| Max | 349 | 700 | 743 | 405 | 702 | 709 | 340 | | |
| CV % | 42% | 41% | 17% | 37% | 39% | 16% | 24% | | |
| GeoMean | 227.1 | 480.2 | 627.5 | 250.6 | 481.3 | 607.9 | 256.2 | | |
| GeoCV % | 46.9% | 49.9% | 16.7% | 35.0% | 44.9% | 15.9% | 24.4% | | |

As illustrated in FIG. 5, antibody systemic exposure was found slightly above dose proportional across dose range from 0.5 to 500 mg/m² (phase 1b study). Mean T_{1/2} were 10 days and 16 days respectively for 250 and 500 mg/m² in the phase 1b study, and the model with QuantPharm showed T1/2 closer to 24 days. The Cₘᵢₙ was set at the average below 3 numbers, which was 130 μg/mL. NTproBNP started to increase 1 week post last dose (880 hrs) when Cₘᵢₙ for 500 mg/m² was dropping ˜100 μg/mL (N=7 pt). Other biomarkers were highly variable.

Half maximal effective concentration (EC50) for highest binding amyloid (in vitro) was ˜157 pug/mL, Michaelis-Menton EC 90 (calculated) was ˜36.5 μg/mL (95 CI 5-134 μg/mL).

Modeling and simulation of the Phase 1a/1b indicated that the minimum predicted steady-state C_{minSS} (among patients from the analysis data set) achieved this level following: 250 mg/m² QW, 750 mg/m² Q2W, or 1000 mg/m² Q3W dosing regimens. With Q4W dosing, even 1000 mg/m² dose achieved only 82.7% target occupancy at C_{minSS} for a patient with the lowest exposure.

The study antibody minimum exposure Cₘᵢₙ (C_{trough}, μg/mL) was evaluated by dose. As shown in Table 5, at 500 mg/m² overall the trough exposures overlapped between Phase 2 and Phase 1b, with Phase 2 trending higher. Between Phase 2 and 1b dose 3/dose 1 accumulation ratio was 2.3 vs. 4.4-fold. In this phase 2 low to moderate variability was observed.

Minimum C_{trough} for 90% receptor occupancy was achieved at >130 μg/mL, after 2nd weekly dose under 1000 mg/m²; 3rd weekly dose under 750 mg/m²; 6th weekly dose under 500 mg/m². The 130 μg/mL target was based on the phase 1a/1b study and may likely change with new Phase 2 data.

TABLE 5

| | Dose 1 | | | Dose 2 | | | Dose 3 | | |
|---|---|---|---|---|---|---|---|---|---|
| | 500 | 750 | 1000 | 500 | 750 | 1000 | 500 | 750 | 1000 |
| Cₘᵢₙ | mg | mg | mg | mg | mg | mg | mg | mg | mg |
| N | 4 | 3 | 6 | 4 | 3 | 3 | 4 | 3 | 3 |
| Mean | 57 | 102 | 119 | 104 | 222 | 324 | 139 | 274 | 353 |
| SD | 8.5 | 43.3 | 75.8 | 31.2 | 124.5 | 37.6 | 35.1 | 130.7 | 66.2 |
| Min | 48 | 55 | 26 | 73 | 91 | 281 | 108 | 127 | 277 |
| Median | 55 | 111 | 113 | 101 | 238 | 340 | 135 | 316 | 388 |
| Max | 68 | 140 | 241 | 141 | 338 | 351 | 179 | 378 | 395 |
| CV % | 15% | 42% | 64% | 30% | 56% | 12% | 25% | 48% | 19% |
| GeoMean | 56.0 | 94.8 | 95.9 | 100.5 | 193.8 | 322.5 | 135.7 | 247.5 | 348.8 |
| GeoCV % | 14.7% | 51.9% | 92.7% | 30.9% | 77.0% | 12.1% | 25.7% | 63.9% | 20.2% |

| | Dose 4 | | | Dose 5 | | | Dose 6 | | |
|---|---|---|---|---|---|---|---|---|---|
| | 500 | 750 | 1000 | 500 | 750 | 1000 | 500 | 750 | 1000 |
| Cₘᵢₙ | mg | mg | mg | mg | mg | mg | mg | mg | mg |
| N | 4 | 3 | 3 | 4 | 2 | 0 | 4 | 2 | 0 |
| Mean | 129 | 330 | 396 | 136 | | 193 | | | |
| SD | 17.9 | 163.2 | 31.3 | 41.3 | | 38.4 | | | |
| Min | 105 | 147 | 360 | 76 | | 139 | | | |
| Median | 132 | 381 | 411 | 152 | | 203 | | | |
| Max | 148 | 461 | 417 | 167 | | 228 | | | |
| CV % | 14% | 49% | 8% | 30% | | 20% | | | |
| GeoMean | 128.3 | 295.6 | 395.1 | 130.4 | | 189.8 | | | |
| GeoCV % | 14.5% | 67.4% | 8.1% | 38.0% | | 22.0% | | | |

The study antibody AUCτ (μg×hr/mL) was evaluated by dose. As shown in Table 6, at 500 mg/m² overall the cumulative exposures were comparable between Phase 2 and Phase 1b studies. There was overlapping AUCτ values after doses 1 and 4. In the phase 2 study, dose 3/dose 1 accumulation ratio was 3.3 to 4 vs. 2-fold in the phase 1b.

In this phase 2 study, there was approximately 100% higher AUCτ after doses 2 and 3 as compared to the phase 1b study; and variability was low to moderate.

TABLE 6

| | Dose 1 | | | Dose 2 | | | Dose 3 | | | Dose 4 | | |
|---|---|---|---|---|---|---|---|---|---|---|---|---|
| | 500 | 750 | 1000 | 500 | 750 | 1000 | 500 | 750 | 1000 | 500 | 750 | 1000 |
| AUC_{τ} | mg | mg | mg | mg | mg | mg | mg | mg | mg | mg | mg | mg |
| N | 4 | 3 | 6 | 4 | 3 | 3 | 4 | 3 | 3 | 4 | 3 | 2 |
| Mean | 15240 | 29879 | 30335 | 25651 | 51159 | 67003 | 32082 | 55333 | 68947 | 62061 | 141780 | |
| SD | 1439 | 14672 | 12592 | 1508 | 22865 | 5049 | 4399 | 23269 | 12331 | 14232 | 60367 | |
| Min | 13773 | 17545 | 13430 | 24286 | 25133 | 62988 | 28302 | 29046 | 54721 | 48016 | 72328 | |
| Median | 14987 | 25986 | 34455 | 25260 | 60324 | 65350 | 31281 | 63660 | 75535 | 62622 | 171357 | |
| Max | 17212 | 46105 | 43881 | 27800 | 68019 | 72671 | 37462 | 73292 | 76585 | 74983 | 181655 | |
| CV % | 9% | 49% | 42% | 6% | 45% | 8% | 14% | 42% | 18% | 23% | 43% | |
| GeoMean | 15190.0 | 27598.5 | 27688.7 | 25618.8 | 46894.7 | 66878.4 | 31859.5 | 51365.2 | 68152.4 | 60818.7 | 131064.9 | |
| GeoCV % | 9% | 52% | 53% | 6% | 59% | 7% | 14% | 53% | 19% | 24% | 55% | |

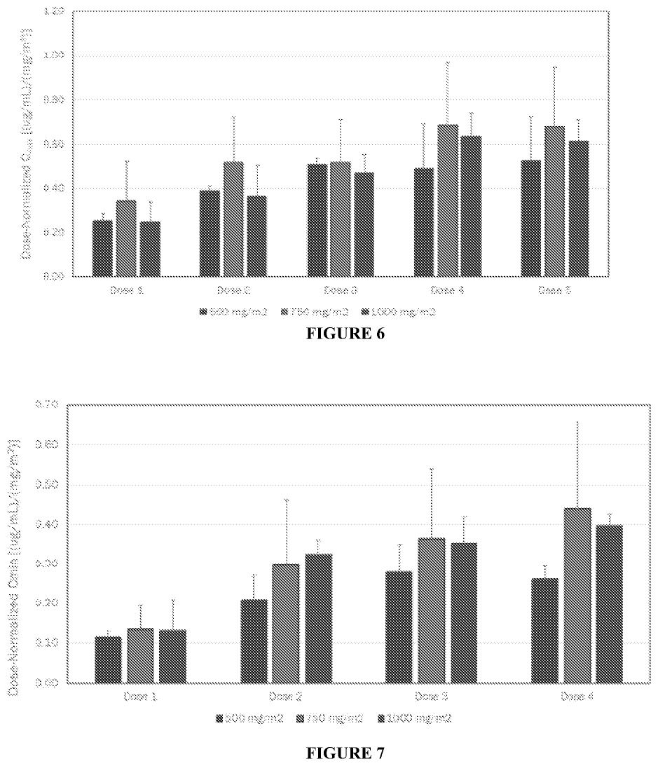

Dose proportionality assessment for Cₘₐₓ/dose was evaluated. As illustrated in FIG. 6 and Table 7, in this phase 2 study, Cₘₐₓ increased approximately dose proportionally from 500 to 1000 mg/m². This was especially the case after dose 3, which indicated saturation of target mediated drug disposition (TMDD) at 750 mg/m².

TABLE 7
Regression Lines

| Dose (N) | m |
|---|---|
| 1 (4, 3, 6) | −0.00002 |
| 2 (4, 3, 6) | −0.00005 |
| 3 (4, 3, 3) | −0.00008 |
| 4 (4, 3, 3) | 0.00030 |
| 5 (4, 3, 3) | 0.00020 |

Further, dose proportionality assessment for Cₘᵢₙ/dose was evaluated. As illustrated in FIG. 7 and Table 8, in this phase 2 study, Cₘᵢₙ increased approximately dose proportionally from 500 to 1000 mg/m². This was especially the case after dose 1, which indicated saturation of target mediated drug disposition (TMDD) at 750 mg/m².

TABLE 8
Regression Lines

| Dose (N) | m |
|---|---|
| 1 (4, 3, 6) | −0.00004 |
| 2 (4, 3, 3) | 0.0002 |
| 3 (4, 3, 3) | 0.0002 |
| 4 (4, 3, 3) | 0.0003 |

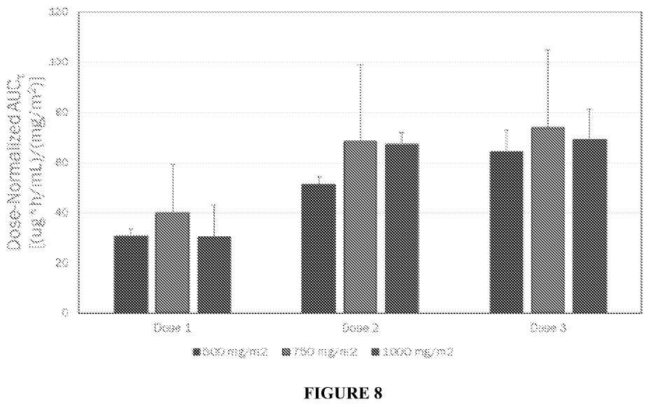

Additionally, dose proportionality assessment for AUCτ/dose was evaluated. As illustrated in FIG. 8 and Table 9, AUCτ increased approximately dose proportionally from 750 to 1000 mg/m², indicating saturation of target mediated drug disposition (TMDD) at 750 mg/m².

TABLE 9
Regression Lines

| Dose (N) | m |
|---|---|
| 1 (4, 3, 6) | −0.00030 |
| 2 (4, 3, 3) | 0.03140 |
| 3 (4, 3, 3) | 0.00960 |

In conclusion, the preliminary data of the PK study of the presently described antibody show that PK exposures increased with increasing doses over the studied dose range of 500 to 1000 mg/m² and that TMDD led to > dose-proportional increase in PK exposures between 500 and 750 mg/m² and approximately dose-proportional increase between 750 and 1000 mg/m². The information supported a dosing with 1000 mg/m², given the maximum tolerated dose with lack of safety concerns, and that all subjects reached > desired Cₘᵢₙ after 2^{nd} dose (immediate, complete, and sustained target saturation). Therefore, this study revealed a P3D (Recommended Phase 3 dose) defined in the Phase 3 study protocols as 4 QW loading dose followed by Q2W maintenance; dose level 1000 mg/m².

The complete gathering of PK and PD data in this Phase 2 at the higher dose levels, the update of PK/PD model integrating Phases 1 and 2 data will be used to refine the Phase 3 optimal dosing regimen and might establish a new target C_{trough}.

While there were limitations of differences in the BA assay for PK, test material, and presence vs. absence of CyBorD (some patients in Phase 1b were treated with chemotherapy before study), antibody exposures following the first dose (Dose 1) overlapped between the two studies but were ˜30-100% higher after subsequent doses for the Phase 2 study.

### Example 3

### Antibody Formulation and Dosage

### Antibody Formulation:

The formulation of the antibody for administration to subject was defined as containing:
30 mg/mL antibody, 10 mL per vial (=300 mg antibody),
25 mM Sodium Acetate,
50 mM Sodium Chloride,
1% Mannitol, and
0.01-0.05% Polysorbate 80.

The formulation was kept at pH 5.5

### Dose Coverage Evaluation:

The study of population pharmacokinetics has shown no correlation with body surface area (BSA) or Weight. The best dose by BSA was determined to be 1000 mg/m², with a second-best option at 750 mg/m², which might want be used as a step down).

The coverage for all doses between 250-1375 mg/m² revealed that the best dose by weight evaluated at 25 mg/kg; the second-best option being 18.75 mg/kg.

The coverage for all doses between 6.25-31.25 mg/kg revealed that the target-mediated drug disposition (TMDD, the phenomenon in which a drug binds with high affinity to its pharmacological target site) occurred at 750 mg/m² and above; which is a critical observation for appropriated dosing across all patient amyloid subtypes and severity levels.

Surprisingly, all patient subtypes and severities had similar PK exposure profiles where it is expected to see potentially difference in clearance and saturation exposures. Low/medium patient variability was observed both with an antibody dose along and in combination with plasma cell directed therapy (PCD).

### Fixed Dose Coverage:

For fixed doses by weight, patients were separated in 3 dose groups receiving 25 mg/kg:
40-70 kgs-1750 mg per dose
71 kg-100 kg-2500 mg/dose
>100 kg-2750 mg/dose
For fixed dose by BSA, patients were separated 3 dose groups:
1.15-1.70 BSA—fixed dose of 1700 mg per dose
1.71-2.40 BSA—fixed dose of 2400 mg per dose
>2.41 BSA—fixed dose of 2750 mg per dose

BSA was calculated by multiple methods, as illustrated in Table 10.

TABLE 10

| Height | Weight (lb) | d&d | G&G | Haycock | Mosteller | | | |
|---|---|---|---|---|---|---|---|---|
| 4′6″ | 77 | 137 | 35 | 1.15 | 1.17 | 1.15 | 1.15 | |
| 5′0″ | 113 | 152 | 50 | 1.45 | 1.47 | 1.46 | 1.45 | |
| 6′2″ | 165 | 188 | 75 | 2 | 1.98 | 1.97 | 1.98 | |
| 5′5″ | 250 | 165 | 113 | 2.17 | 2.31 | 2.33 | 2.28 | |
| 5′9″ | 264 | 178 | 120 | 2.32 | 2.45 | 2.47 | 2.42 | |
| 5′4″ | 170 | | 163 | 1.83 | 1.83 | 1.89 | 1.89 | 1.87 |
| | (average) | | | | | | | |
| 5′9″ | 200 | | 175 | 2.07 | 2.07 | 2.12 | 2.13 | 2.10 |
| | (average) | | | | | | | |

### Loading Dose Regimen:

C_{trough} levels covered 30 μg/mL-400 μg/mL.

It was determined by binding numbers from 5 amyloid subtypes from liver heart and spleen. NTproBNP biomarker data from Phase 1b (100 μg/mL) were used, with GLS data (doses >100 mg/m² had effect): >30 μg/mL.

It was established that a loading dose to rapidly achieve C_{trough} of 130 μg/mL could be achieved by:
1 dose for 1500 mg/m²,
2 qw or 3 qw doses for 1000 mg/m² or 25 mg/kg,
4 qw doses for 750 mg/m² or 18.75 mg/kg,
6 qw doses for 500 mg/m² or 12.5 mg/kg, which covered 4 qw doses for all dose levels including fixed doses.

### Maintenance Dose Regimen

The maintenance dose regimen were established to:
cover q2w doses for all doses BSA and weight based
cover q4w for 1375 mg/m²
cover q3w and q4w for 1000 mg/m² or 25 mg/kg
cover q3w for 750 mg/m² or 18.75 mg/kg
   Dose Regimen in Combination with PCD

It was further established that the antibody can be safely dosed in combination with no impact on exposure. It was found ideal to dose antibody first and to modify the dose based on maintaining normal neutrophils and monocytes.

The hypothesis behind this was that a better organ response can be obtained by combination dosing of the antibody and doxycycline based on the mechanism of action. The antibody removes toxic light chains, protofibrils, fibrils and amyloid, and amyloid builds up in organs with extracellular matrix, while doxycycline inhibits matrix metalloproteases and prevents extracellular matrix allows greater access of the antibody to amyloid and N-terminus epitope.

The CARES clinical program consists of two parallel double-blind, randomized, event-driven global Phase 3 studies, which are evaluating the efficacy and safety of the antibody in AL amyloidosis patients who are newly diagnosed with AL amyloidosis and naïve to standard of care (SoC) treatment (cyclophosphamide-bortezomib-dexamethasone (CyBorD) chemotherapy). One study is enrolling approximately 267 patients with Mayo stage IIIa disease [the antibody+CyBorD (n=178) and placebo+CyBorD (n=89)] and one study is enrolling approximately 111 patients with Mayo stage IIIb disease [the antibody+CyBorD (n=74) and placebo+CyBorD (n=37)]. The studies will be conducted at approximately 70 sites across North America, the United Kingdom, Europe, Israel, Japan, and Australia. In each study, participants are being randomized in a 2:1 ratio to receive either the antibody plus SoC or placebo plus SoC once weekly for four weeks. This will be followed by a maintenance dose administered every two weeks until a minimum of 54 deaths for Study 1 and 77 deaths for Study 2 (a minimum treatment duration of 12 months is expected). Patients will continue follow-up visits every 12 weeks. The primary study objectives are overall survival and the safety and tolerability of the antibody. Key secondary objectives assess functional improvement in the six-minute walk test (6MWT), quality of life measures (Kansas City Cardiomyopathy Questionnaire Overall Score & Short Form 36 version 2 Physical Component Score) and cardiac improvement (Global Longitudinal Strain, or GLS).

Patient baseline characteristics and demographics are presented in Table 11. At least 9/13 (69.2%) patients in Study 1 and 23/39 (59%) patients in Study 2 have received at least 4 doses of CAEL-101 concurrently with anti-PCD therapy.

These ongoing trials will evaluate the efficacy and safety of CAEL-101 as first-in-class treatment to reduce amyloid burden in patients with cardiac AL-A. Notably, Study 1 (Mayo Stage IIIb) is the first randomized, placebo-controlled efficacy clinical trial to formally assess the effects of a pharmacological in this severely ill population. Because the median expected survival for Mayo Stage IIIb patients is far shorter than for Mayo Stage IIIa patients, the resulting sample size required for the Mayo Stage IIIB study is less (111 patients) than for the Mayo Stage IIIA study (267 patients). Importantly, these studies include patients identified as Stage III and IV based on the 2012 Mayo staging system (Kumar, S. et al., J. Clin. Oncol., 30:989-95, 2012).

TABLE 11
Table 11: Patient baseline characteristics and demographics

| | | Study 1 | Study 2 |
|---|---|---|---|
| Parameter | | (Mayo Stage IIIb) | (Mayo Stage IIIa) |
| Number of patients enrolled to date | | 13 | 39 |
| Age | N | 11 | 35 |
| | Mean (SD), y | 69.2(6.37) | 65.6(7.41) |
| Sex, n (%) | Male | 7(53.8) | 23(59.0) |
| | Female | 5(38.5) | 12(30.8) |
| | Missing | 1(7.7) | 14(10.3) |
| Race | Black or African | 1(7.7) | 3(7.7) |
| | American | | |
| | White or Caucasian | 7(53.8) | 25(64.1) |
| | Asian | 1(7.7) | 5(12.8) |
| | Other | 2(15.4) | 1(2.6) |
| | Missing | 2(15.4) | 5(12.8) |
| Ethnicity, | Hispanic or Latino | 0(0.0) | 1(2.6) |
| (n %) | Not Hispanic or Latino | 9(69.2) | 30(76.9) |
| NT-proBNP | N | 9 | 30 |
| | Median (range), pg/mL | 12,800 (8940-35,418) | 3972.5 (962-7768) |
| eGFR | N | 6 | 22 |
| | Median (range), | 49.5 (29-89) | 69.1 (33-105) |
| | mL/min/173 m² | | |
| Proteinuria | N | 9 | 26 |
| | Median (range), g/24 h | 0.096 (0.0321-14.879) | 0.026 (0.0001-12.240) |

Abbreviations: eGFR, estimated glomerular filtration rate;
NT-proBNT, N-terminal pro-brain natriuretic peptide;
SD, standard deviation

### Example 4

Safety and Tolerability of Antibody in Combination with Anti-Plasma Cell Dyscrasia Therapy in Patients with Al Amyloidosis: 1-Year Results from an Open-Label Phase 2 Trial

Light-chain (AL) amyloidosis is a rare, systemic disease caused by plasma cell dyscrasia (PCD). Excess immunoglobulin light chains misfold and form insoluble amyloid fibrils that deposit in organs, primarily the heart. Survival depends largely on the extent of heart involvement. Current therapies target PCD to halt fibril formation but do not treat existing fibril deposited in organs. In a Phase 2 trial, patients were treated with weekly infusions of up to 1000 mg/m² of the antibody, combined with anti-PCD therapy as standard of care (SOC), demonstrating this dose was well tolerated and appropriate for Phase 3. The long-term safety and tolerability of CAEL-101, administered with SOC was evaluated.

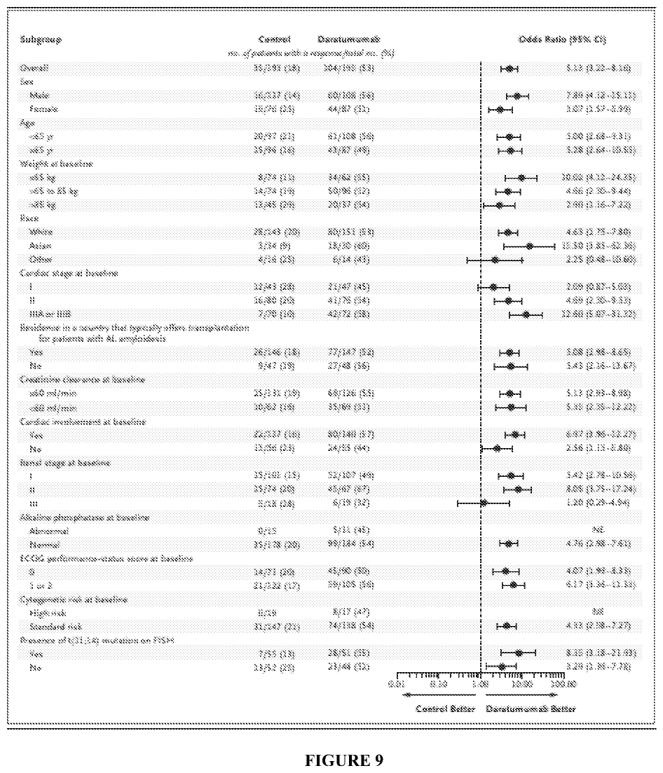

Adult patients with confirmed AL amyloidosis diagnosis (Mayo Stages I, II, IIIa), 6 month minimum life expectancy, and measurable hematologic disease were eligible for this ongoing, open-label, phase 2 study (NCT04304144). Patients with MM, supine systolic blood pressure <90 mm Hg, or symptomatic orthostatic hypotension were excluded. All patients received the antibody 1000 mg/m² every other week with SOC anti-PCD therapy until investigator decided anti-PCD was no longer needed (FIG. 9). Safety assessments included treatment-emergent adverse events (TEAEs), clinical laboratory tests, electrocardiograms, vital signs, and physical examinations. Pharmacokinetic endpoints included maximum serum concentration (Cₘₐₓ) and minimum serum concentration of CAEL-101 prior to next dose (C_{trough}). Exploratory endpoints included biomarkers for cardiac function (cardiac troponin T [cTnT] and N-terminal pro-brain natriuretic peptide [NT-proBNP]), renal function (estimated glomerular filtration rate [eGFR] and proteinuria) and changes in free light chain (FLC). There were also immunogenicity assessments.

Initial long-term results were assessed when some of the patients have been treated for a year. At that point, the 25 patients averaged 65.2 years (range 47 to 80), with the majority male (72.0%). Mayo Stages I (8.0%), II (76.0%), and IIIa (16.0%) reflected the wide range of disease severity in enrolled patients; 20 (80.0%) presented with cardiac involvement, 9 (36.0%) with renal involvement, and 20 (80.0%) had received prior anti-PCD therapy. Twenty-four (96.0%) patients experienced TEAEs, but only 6 (24.0%) experienced a possibly treatment related TEAE (Table 12). Eight (32.0%) patients experienced at least 1 Grade ≥3 TEAE and 7 (28.0%) experienced at least 1 serious adverse event. There were 3 (12.0%) discontinuations; 1 death due to septic pneumonia (investigator determined not related to the antibody), one heart transplant, and one patient who withdrew consent. Most common TEAEs included nausea (9 [36.0%]], constipation (8 [32.0%]), and diarrhea, fatigue, or rash (7 [28.0%] each).

TABLE 12
Table 12: Summary of Most Common TEAEs

| | Antibody + Anti-PCD |
|---|---|
| MedDRA Preferred Term | (N = 25) |
| Patients with ≥ 1 TEAE | 24(96.0%) |
| Patients with ≥ 1 possible TEAE | 6(24.0%) |
| related to treatment | |
| Patients with ≥ 1 TEAE of Grade ≥ 3 | 8(32.0%) |
| Patients with ≥ 1 SAE | 7(28.0%) |
| Discontinuations | 3(12.0%) |
| Deaths | 1(4.0%) |
| Nausea | 9(36.0%) |
| Constipation | 8(32.0%) |
| Diarrhea | 7(28.0%) |
| Fatigue | 7(28.0%) |
| Rash | 7(28.0%) |

Addition of daratumumab (n=12) to the anti-PCD combination treatment of cyclophosphamide-bortezomib-dexamethasone (CyBorD) did not alter the pharmacokinetic or tolerability profile of the antibody. There are 20 cardiac evaluable patients. Overall, 18 out of 20 (90%) current cardiac evaluable patients (baseline NT-proBNP ≥332 ng/L and ≥1 post-first-dose NT-proBNP value), showed improvement or were stable at the last evaluable timepoint. 7 (35.0%) have responded (≥30% NT-proBNP decrease from baseline) and 11 (55%) are stable from baseline (±30% change from baseline). Renal evaluable patients, as determined by Investigator at a single site, showed a similar proteinuria response. There are 9 renal evaluable patients. Overall, 8 out of 9 (88.9%) current patients with renal impairment at baseline showed a renal response (determined by investigator at a single site). Renal response was defined as ≥30% decrease in proteinuria following treatment. In 3 patients, proteinuria decreased to <0.5 g/24 h.

This ongoing trial is evaluating the long-term safety and tolerability of the antibody administered with anti-PCD SOC as a treatment to reduce amyloid burden in patients with cardiac AL amyloidosis. The antibody was well tolerated when administered with anti-PCD therapy. Most TEAEs observed were mild to moderate in severity and did not require intervention. There were no meaningful differences in tolerability or exposure to the antibody when daratumumab was added to the anti-PCD regimen. Improvements in cardiac and renal response biomarkers were observed in most patients presenting with cardiac or renal involvement, respectively, at study entry.

After approximately 1-year, the antibody, as part of an AL amyloidosis treatment strategy, demonstrates to be well tolerated. This updated report confirms previous findings for the use of the antibody in combination with anti-PCD.

Safety, tolerability, and biomarker data were reassessed after all patients with AL amyloidosis enrolled in the trial received 1 year of treatment with CAEL-101, administered initially with cyclophosphamide-bortezomib-dexamethasone (CyBorD)+daratumumab.

CAEL-101≤1000 mg/m² was administered every other week with anti-PCD therapy as directed by the investigator. In addition to safety assessments, cardiac and renal response were assessed by change over time in N-terminal pro-brain natriuretic peptide (NT-proBNP) and proteinuria, respectively.

Patients (n=25; mean age 65 years; 72% male) with Mayo Stage I (8%), II (76%), and IIIa (16%) AL amyloidosis were treated with CAEL-101 for 1 year at this analysis. Patients presented with cardiac involvement (n=22), renal involvement (n=9, reported from one site), and prior anti-PCD therapy (n=20). All 25 patients experienced treatment-emergent adverse events (TEAEs), and 6 (24%) experienced possibly treatment-related TEAEs. Five patients discontinued for non-treatment-related reasons (Table 13). Fifteen (60%) patients experienced TEAEs of Grade ≥3 severity and 13 (52%) experienced ≥1 serious adverse event (SAE). The most common TEAEs were nausea (n=10), constipation or fatigue (n=9 each), anemia, insomnia, or diarrhea (n=8 each), and dizziness, cough, or rash (n=7 each).

As illustrated in FIG. 11, after all of the enrolled patients had received 1 year of CAEL-101 treatment of the 22 cardiac evaluable patients, 10 (46%) patients experienced ≥30% NT-proBNP decrease from baseline, 4 (18%) were stable (±30% change from baseline), and 3 (14%) showed disease progression (≥30% NT-proBNP increase from baseline); data for 5 patients were missing. Of the 9 renal evaluable patients, 8 showed ≥30% decrease from baseline in proteinuria.

The long-term safety evaluation of CAEL-101 continues in this study. All patients currently enrolled have been treated for at least 1 year. At this 1-year time point, CAEL-101 has been generally well-tolerated without evidence of organ toxicity. Organ response persisted even after cessation of anti-PCD treatment. Most TEAEs were mild to moderate. A Phase 3 program has commenced to elucidate the efficacy and safety of CAEL-101 in cardiac AL amyloidosis European Modification Mayo Stages IIIa and IIIb.

TABLE 13
Summary of Treatment-Emergent Adverse Events and Discontinuations
After One Year of CAEL-101 Treatment

| | CAEL-101 + Anti-PCD |
|---|---|
| Summary | Treatment (N = 25) |
| Patients with ≥ 1 TEAE | 25 (100%) |
| Patients with ≥ 1 TEAE possibly | 6 (24%) |
| treatment related | |
| Patients with ≥ 1 TEAE of Grade ≥ 3 | 15 (60%) |
| Patients with ≥ 1 SAE | 13 (52%) |
| Treatment-related discontinuations | 0 (0%) |
| of CAEL-101 | |
| Total discontinuations | 5 (25%) |
| Death due to septic pneumonia | 1 (4%) |
| Heart transplant | 1 (4%) |
| Heart and kidney transplant | 1 (4%) |
| Physician decision | 1 (4%) |
| Withdrawal of consent | 1 (4%) |
| MedDRA Preferred Term | |
| Nausea | 10 (40%) |
| Constipation | 9 (36%) |
| Fatigue | 9 (36%) |
| Anemia | 8 (32%) |
| Diarrhea | 8 (32%) |
| Insomnia | 8 (32%) |
| Dizziness | 7 (28%) |
| Cough | 7 (28%) |
| Rash | 7 (28%) |

### Example 5

### Organ Response of the Antibody

Administration of the disclosed antibody showed some improvement of kidney function. 7 patients with kidney involvement and all had organ responses.

In particular, one patient with partial response (PR) subsequently progressed back to stable disease (SD). Despite this, the patient had an ongoing deepening renal organ response currently showing a 76% reduction in 24-hour proteinuria without change in anti-plasma cell therapy. The median was 56 days to organ response.

Administration of the disclosed antibody showed some Cardiac Response. 3/8 evaluable patients were newly diagnosed and their NT-proBNP has shown increases in the first 3 months of CyBorD therapy. One of 8 patients achieved cardiac organ response by NT-proBNP criteria.

### Example 6

The disclosed antibody dosed at 1000 mg/m² is the recommended dose in combination with CyBorD for the ongoing randomized, double blind Phase 3 trials described above. Organ responses particularly in the kidney were common even in relapsed patients. Only 1 patient is no longer on study due to need for change in anti-plasma cell therapy. Significantly, organ responses have been seen even without ongoing hematologic (partial response) PR.

### Example 7

### Daratumumab-Based Treatment for Immunoglobulin Light-Chain Amyloidosis

In this phase 3 trial involving patients with newly diagnosed AL amyloidosis, subcutaneous daratumumab in combination with bortezomib, cyclophosphamide, and dexamethasone resulted in a significantly higher frequency of a hematologic complete response than bortezomib, cyclophosphamide, and dexamethasone alone. Hematologic responses were deeper and occurred more rapidly in the daratumumab group.

All the patients received subcutaneous bortezomib at a dose of 1.3 mg per square meter of body-surface area, cyclophosphamide at a dose of 300 mg per square meter orally or intravenously (500 mg maximum weekly dose), and dexamethasone at a dose of 40 mg orally or intravenously once weekly for six cycles of 28 days each. For patients who were older than 70 years of age, were underweight (body-mass index [the weight in kilograms divided by the square of the height in meters], <18.5), or had hypervolemia, poorly controlled diabetes mellitus, or previous unacceptable side effects associated with glucocorticoid therapy, dexamethasone could be administered at a dose of 20 mg weekly at the discretion of their physician. Patients who were assigned to the daratumumab group received 1800 mg of daratumumab per 15 mL administered subcutaneously, co-formulated with recombinant human hyaluronidase PH20, weekly in cycles 1 and 2, every 2 weeks in cycles 3 through 6, and every 4 weeks thereafter until disease progression, the start of subsequent therapy, or for a maximum of 24 cycles from the start of the trial, whichever occurred first.

A total of 388 patients (195 in the daratumumab group and 193 in the control group) underwent randomization. The demographic and clinical characteristics of the patients at baseline were balanced between the groups (Table 14). The median age was 64 years (range, 34 to 87), and the median time since diagnosis was 43 days (range, 5 to 1611). The median baseline difference between the involved and uninvolved free light-chain levels was 187 mg per liter (range, 1 to 9983). A total of 254 patients (65.5%) had two or more organs involved; 71.4% of the patients had heart involvement, and 59.0% had kidney involvement. The majority of patients (76.8%) were classified as having a cardiac stage of II or higher. Among the 388 patients who underwent randomization, 381 (193 in the daratumumab group and 188 in the control group) received at least one dose of trial treatment. At the time of clinical data cutoff for the primary analysis (Feb. 14, 2020), a total of 52 patients (26.9%) in the daratumumab group and 68 patients (36.2%) in the control group had discontinued the intervention before the protocol-defined completion of treatment. In the control group, 121 patients (64.4%) received six cycles of treatment as specified by the protocol. In the daratumumab group, 159 patients (82.4%) completed six cycles of trial treatment, and 149 (77.2%) continued single-agent subcutaneous daratumumab after completing the first six treatment cycles; at the time of analysis, 141 of 195 patients (72.3%) were continuing to receive daratumumab. Dose reductions were similar in the daratumumab group and the control group (cyclophosphamide, 17.6% and 13.8%, respectively; bortezomib, 25.9% and 19.7%; dexamethasone, 27.5% and 27.7%; daratumumab dose reductions were not permitted). The median duration of therapy was 9.6 months in the daratumumab group and 5.3 months in the control group.

TABLE 14
Table 14. Demographic and Disease Characteristics of the
Patients at Baseline (Intention-to-Treat Population).

| | | Daratumumab group | Control group |
|---|---|---|---|
| | | (N = 195) | (N = 193) |
| Age | Median (range) - yr | 62 (34-87) | 64 (35-86) |
| | Distribution - no. (%) | | |
| | <65 yr | 108 (55.4) | 97 (50.3) |
| | >65 yr | 87 (44.6) | 96 (49.7 |
| Sex - no(%) | Male | 108 (55.4) | 117 (60.6) |
| | Female | 87 (44.6) | 76 (39.4) |
| Eastern Cooperative | 0 | 90 (46.2) | 71 (36.8) |
| Oncology Group | 1 | 86 (44.1) | 106 (54.9) |
| (ECOG) performance | 2 | 19 (9.7) | 16 (8.3) |
| status score - no (%) | | | |
| Involved organs | Median(range) | 2 (1-5) | 2 (1-6) |
| | Distribution - no (%) | | |
| | Heart | 23 (11.8) | 137 (71.0) |
| | Kidney | 10 (5.1) | 114 (59.1) |
| | Liver | 15 (7.7) | 16 (8.3) |
| | Other | 127 (65.1) | 124 (64.2) |
| Cardiac stage-no (%) | I | 47 (24.1) | 43 (22.3) |
| | II | 76 (39.0) | 80 (41.5) |
| | IIIA | 70 (35.9) | 64 (33.2) |
| | IIIB | 2 (1.0) | 6 (3.1) |
| Renal Stage - no/total no (%) | I | 107/193 (55.4) | 101/193 (52.3) |
| | II | 67/193 (34.7) | 74/193 (38.3) |
| | III | 19/193 (9.8) | 18/193 (9.3) |
| Creatine clearance - no(%) | <60 ml/min | 69 (35.4) | 62 (32.1) |
| | ≥60 ml/min | 126 (64.6) | 131 (67.9) |
| Median NT-proBNP level | | 1388.6 (51-10,182) | 1746.0 (51-12,950) |
| (Range) - ng/liter | | | |
| Median estimated GFR | | 77.8 (21-126) | 76.2 (20-121) |
| (range) - ml/min/1.73 m² | | | |

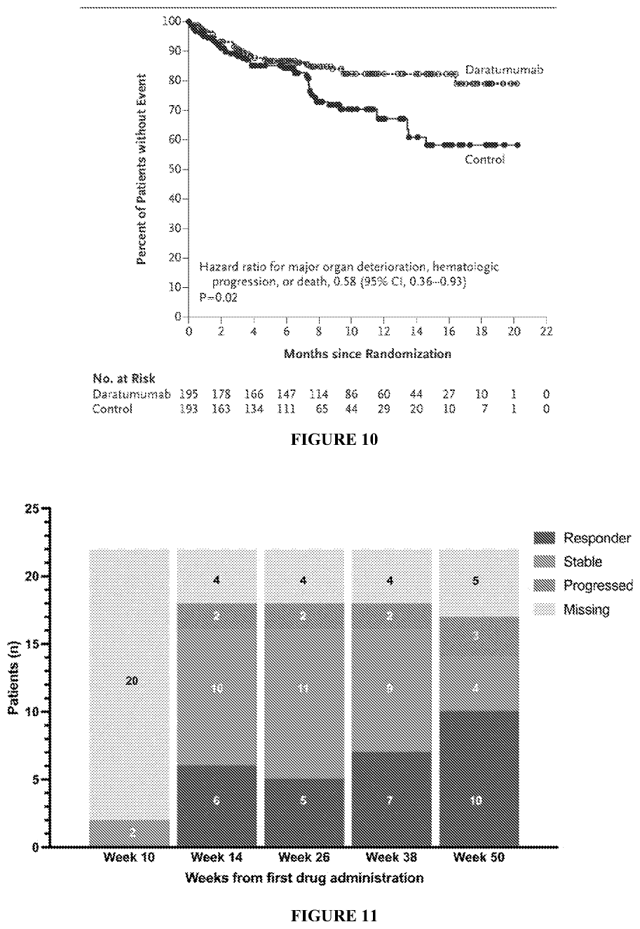

With a median follow-up of 11.4 months (range, 0.03 to 21.3), 104 patients (53.3%) in the daratumumab group and 35 patients (18.1%) in the control group had a hematologic complete response (Table 15). This difference was significant (relative risk ratio, 2.9; 95% confidence interval [CI], 2.1 to 4.1; odds ratio, 5.1; 95% CI, 3.2 to 8.2; P<0.001 for both comparisons). The percentages of patients with a hematologic complete response in prespecified subgroups showed consistent benefit in the daratumumab group (Figure. 9). Landmark analysis of hematologic complete response at 6 months showed percentages consistent with overall hematologic complete response (49.7% in the daratumumab group vs. 14.0% in the control group; relative risk ratio, 3.5; 95% CI, 2.4 to 5.2; odds ratio, 6.1; 95% CI, 3.7 to 10.0; P<0.001 for both comparisons). The median time to hematologic complete response was 60 days in the daratumumab group and 85 days in the control group. The percentage of patients who had a hematologic very good partial response or better was 78.5% in the daratumumab group and 49.2% in the control group (relative risk ratio, 1.6; 95% CI, 1.4 to 1.9; odds ratio, 3.8; 95% CI, 2.4 to 5.9). An involved free light-chain level of 20 mg or less per liter was observed more frequently among patients in the daratumumab group than among those in the control group (70.5% vs. 20.2%); similar outcomes were observed for a difference between the involved and uninvolved free light-chain levels of less than 10 mg per liter (63.3% vs. 29.5%) (Table 15). Among patients who could be evaluated for cardiac response (118 in the daratumumab group and 117 in the control group), the percentage who had a cardiac response at 6 months was 41.5% in the daratumumab group and 22.2% in the control group (Table 15); cardiac progression at 6 months was observed in 2.5% and 7.7% of the patients, respectively. Among patients who could be evaluated for renal response (117 in the daratumumab group and 113 in the control group), the percentage who had a renal response at 6 months was 53.0% in the daratumumab group and 23.9% in the control group (Table 15); renal progression at 6 months was observed in 4.3% and 11.5% of the patients, respectively. Survival free from major organ deterioration or hematologic progression was longer in the daratumumab group than in the control group (hazard ratio for major organ deterioration, hematologic progression, or death, 0.58; 95% CI, 0.36 to 0.93; P=0.02) (FIG. 10). Hematologic progression occurred in 8 patients (4.1%) in the daratumumab group and in 25 patients (13.0%) in the control group. Survival free from major organ deterioration, hematologic progression, or subsequent treatment was also longer in the daratumumab group than in the control group (hazard ratio for major organ deterioration, hematologic progression, subsequent treatment, or death, 0.39; 95% CI, 0.27 to 0.56).

TABLE 15
Table 15: Summary of Overall Confirmed Hematologic Responses
and Cardiac and Renal Responses at 6 Months

| | | Daratumumab Group | Control Group |
|---|---|---|---|
| Response | | (N = 195) | (N = 193) |
| Hematologic | | | |
| response | | | |
| Any response | no. of patients | 179 | 148 |
| | Percent of patients (95% CI) | 91.8 (87.0-95.2) | 76.7 (70.1-82.5) |
| Complete | no. of patients | 104 (P value < 0.001) | 35 (P value < 0.001) |
| response | Percent of patients (95% CI) | 53.3 (46.1-60.5) | 18.1 (13.0-24.3) |
| Very good partial response or better - no. (%) | | 153 (78.5) | 95 (49.2) |
| Very good partial response - no. (%) | | 49 (25.1) | 60 (31.1) |
| Partial response - no. (%) | | 26 (13.3) | 53 (27.5) |
| No response - no. (%) | | 8 (4.1) | 38 (19.7) |
| Progressive disease - no. (%) | | 0 | 0 |
| Response could not be evaluated - no. (%) | | 8 (4.1) | 7 (3.6) |
| Cardiac | No. of patients who could | 118 | 117 |
| response at | be evaluated | | |
| 6 months | Percent with a response | 41.5 (32.5-51.0) | 22.2 (15.1-30.8) |
| | (95% CI) | | |
| Renal | No. of patients who could | 117 | 113 |
| response at | be evaluated | | |
| 6 months | Percent with a response | 53.0 (43.5-62.3) | 23.9 (16.4-32.8) |
| | (95% CI) | | |

A total of 19 of 193 patients (9.8%) in the daratumumab group and 79 of 188 patients (42.0%) in the control group received non-cross-resistant subsequent therapy. Of the 79 patients in the control group who received non-cross-resistant subsequent therapy, 48 (61%) received intravenous daratumumab as monotherapy or in combination with other therapies. A total of 13 of 193 patients (6.7%) in the daratumumab group and 20 of 188 patients (10.6%) in the control group received subsequent autologous stem-cell transplantation. Overall survival did not differ substantially between the two groups at the time of this analysis.

The results show that patients with newly diagnosed AL amyloidosis, the addition of daratumumab to bortezomib, cyclophosphamide, and dexamethasone was associated with higher frequencies of hematologic complete response and survival free from major organ deterioration or hematologic progression. Hematologic responses were deeper and occurred more rapidly in the daratumumab group. In this prospective, randomized trial involving patients with newly diagnosed AL amyloidosis, the addition of subcutaneous daratumumab to bortezomib, cyclophosphamide, and dexamethasone resulted in significantly better outcomes.

### Example 8

### Phase 3 Study

Table 16 provides Demographics and Baseline Characteristics of a further Phase III study to assess dosage for the CAEL-101 antibody disclosed herein having a heavy chain variable domain (VH) as set forth in SEQ ID NO:1 and a light chain variable domain (VL) as set forth in SEQ ID NO:2. The results show the range (min/max) and the median doses, e.g., the median dose was 25.6 mg/kg, ranging from 19.8 to 31.1 mg/kg, with the vast majority of patients receiving between 22 and 28 mg/kg.

TABLE 16

| | Stage IIIB | Stage IIIA | Total \| |
|---|---|---|---|
| | (N = 16) | (N = 52) | (N = 68) |
| Parameters | n (%) | n (%) | n (%) |
| Age (yrs) at consent | | | |
| n | 15 | 48 | 63 |
| Mean (SD) | 66.3(10.52) | 60.6(17.78) | 62.0(16.44) |
| Median | 68.0 | 65.5 | 66.0 |
| Min, Max | 36.0, 78.0 | 0.0, 81.0 | 0.0, 81.0 |
| Sex - n (%) | | | |
| Male | 8 (50.0) | 32 (61.5) | 40(58.8) |
| Female | 7 (43.8) | 16(30.8) | 23(33.8) |
| Missing | 1 (6.3) | 4 (7.7) | 5 (7.4) |
| Race - n (%) | | | |
| Black or African | 1(6.3) | 4 (7.7) | 5 (7.4) |
| American | | | |
| White or Caucasian | 11 (68.8) | 35 (67.3) | 46 (67.6) |
| Asian | 1 (6.3) | 7 (13.5) | 8 (11.8) |
| Other | 2 (12.5) | 2 (3.8) | 4 (5.9) |
| Missing | 1 (6.3) | 4 (7.7) | 5 (7.4) |
| Ethnicity - n (%) | | | |
| Hispanic or Latino | 1 (6.3) | 1 (1.9) | 2 (2.9) |
| Not Hispanic or Latino | 13 (1.3) | 43 (82.7) | 56 (82.4) |
| Unknown | 0 (0.0) | 1 (1.9) | 1 (1.5) |
| Not Reported | 1 (6.3) | 3 (5.8) | 4 (5.9) |
| Missing | 1 (6.3) | 4 (7.7) | 5 (7.4) |
| Child-bearing | | | |
| potential - n (%) | | | |
| No | 14 (87.5) | 48 (92.3) | 62 (91.2) |
| Yes | 1 (6.3) | 0 (0.0) | (1.5) |
| Missing | 1 (6.3) | 4 (7.7) | 5 (7.4) |
| Parafin Embedded Tissue | | | |
| Sample Avaiable - n (%) | | | |
| No | 11 (68.8) | 37 (71.2) | 48 (70.6) |
| Yes | 3 (18.8) | 9 (17.3) | 12 (17.6) |
| Missing | 2 (12.5) | 6 (11.5) | 8 (11.8) |
| Weight (kg) | | | |
| n | 15 | 48 | 63 |
| Mean (SD) | 72.6 (13.54) | 72.1(13.79) | 72.2(13.62) |
| Median | 71.4 | 71.0 | 71.0 |
| Min, Max | 47.3, 95.9 | 46.0, 111.6 | 46.0, 111.6 |
| Body Surface Area (m^{A}2) | | | |
| n | 16 | 52 | 68 |
| Mean (SD) | 1.8(0.20) | 1.8 (0.21) | 1.8 (0.21) |
| Median | 1.8 | 1.8 | 1.8 |
| Min, Max | 1.4, 2.2 | 1.4, 2.3 | 1.4, 2.3 |
| Calculated Dose Level (mg) | | | |
| n | 16 | 52 | 68 |
| Mean (SD) | 1841(202.8) | 1833(209.6) | 1835(206.5) |
| Median | 1800 | 1825 | 1815 |
| Min, Max | 1440, 2150 | 1420, 2270 | 1420, 2270 |
| Dose Level (mg/kg) | | | |
| n | 15 | 48 | 63 |
| Mean (SD) | 25.8 (2.22) | 25.7 (2.12) | 25.7 (2.13) |
| Median | 25.5 | 25.7 | 25.6 |
| Min, Max | 22.4, 30.4 | 19.8, 31.1 | 19.8, 31.1 |
| Volume (mL) | | | |
| n | 16 | 52 | 68 |
| Mean (SD) | 61.4 (6.76) | 61.1 (6.99) | 61.2 (6.88) |
| Median | 60.0 | 60.8 | 60.5 |
| Min, Max | 48.0, 71.7 | 47.3, 75.7 | 47.3, 75.7 |

### Example 9

Phase 3 Trials Assessing the Antibody in Patients with Mayo Stage IIIa or Stage IIIB AL Amyloidosis

The Cardiac Amyloid Reaching for Extended Survival (CARES) study design was a two placebo-controlled, double-blind, randomized, international phase 3 trial assessing the antibody disclosed herein in patients with Mayo stage IIIa or stage IIIb AL Amyloidosis. The antibody having a heavy chain variable domain (VH) as set forth in SEQ ID NO:1 and a light chain variable domain (VL) as set forth in SEQ ID NO:2. The aim of this study was to determine whether the antibody with treatment for PCD improves overall survival in patients with Mayo Stage IIIb (NCT04504825; Study 301) or IIIa AL amyloidosis (NCT04512235; Study 302) who are treatment naïve compared to treatment for PCD alone.

For background, AL amyloidosis is a rare, severe, progressive, systemic disorder resulting from the overproduction of amyloidogenic immunoglobulin (Ig) light chains by monoclonal plasma cell dyscrasia (PCD). These amyloidogenic light chains misfold and aggregate into insoluble amyloid fibrils that deposit in multiple organs leading to progressive organ dysfunction/damage and death. The prognosis of patients with AL amyloidosis depends on size of the plasma cell clone and the amyloid burden in tissues, especially in the heart. Patients with extensive cardiac involvement, characterized by high levels of cardiac troponin T (cTnT) and N-terminal pro-brain natriuretic peptide (NT-proBNP), have a poor prognosis, for example. Median survival is 24 months and 4 months for patients in Mayo Stage IIIa and IIIb AL amyloidosis, respectively. For most patients, standard of care (SoC) is anti-PCD therapy to suppress pathological plasma cell proliferation, halt generation of amyloidogenic free light chains, and prevent deposition of new amyloid fibrils and further organ decline. However, a critical need exists for therapies that facilitate the removal of already deposited fibrils and restore organ function. Anti-fibril agents are designed to promote degradation of amyloid fibrils, thereby reducing tissue amyloid burden and improving overall survival and quality of life (QoL).

The antibody described herein in a monoclonal antibody that binds to misfolded Ig light chains in amyloid fibrils and is designed to remove fibrils from tissues and organs. As stated in other examples, CAEL-101 (with and without concurrent anti-PCD SoC) is generally well tolerated up to 1000 mg/m² in phase 1 and 2 trials. Phase 2 data (NCT04304144) indicate that long-term use (up to a median of 49 weeks) of CAEL-101 in combination with cyclophosphamide-bortezomib-dexamethasone (CyBorD) and concurrent use with CyBorD and daratumumab is generally well tolerated. Assessment of cardiac and renal biomarkers in some patients with cardiac and renal impairment at baseline suggests improvements in cardiac and renal disease.

TABLE 17

| Key Inclusion Criteria | Key Exclusion criteria |
|---|---|
| Study 301: AL amyloidosis Stage | Other forms of amyloidosis |
| IIIb with NT-proBNP > 8500 ng/L | Prior treatment for multiple |
| | myeloma |
| Study 302: AL amyloidosis Stage | Multiple myeloma |
| IIIa with NT-proBNP ≥ 650 ng/L | |
| and ≤ 8500 ng/L | |
| Up to 2 weeks of anti-PCD treatment | |
| allowed prior to randomization | |
| Measurable hematologic disease | |
| Histopathological diagnosis | |
| of AL amyloidosis | |
| Cardiac involvement | |
| Adequate bone marrow reserve | |
| Adequate heptic and renal function | |

Patients were treatment-naïve adults with AL amyloidosis Stage IIIb or IIIa (based on the 2013 European Modification of 2004 Mayo Staging) for whom planned first-line treatment for PCD is a CyBorD-based regimen administered as SoC are eligible (Table 18). An additional criterion of high-sensitivity troponin T (hs-TnT) at a threshold of 50 ng/L was included.

TABLE 18

| | | | cTnT or |
|---|---|---|---|
| | | NT-proBNP | hs-Tnt^{a} |
| Stage | Criteria | (pg/mL) | (ng/L)^{b} |
| I | Both markers below | <332 | ≤35.0/50 |
| | the threshold | | |
| II | Either marker above | ≥332 | ≥35.0/50 |
| | the threshold | | |
| IIIa | Both markers above | ≥332 but ≤ 8500 | ≥35.0/50 |
| | the threshold | | |
| IIIb | Both markers above | >8500 | ≥35.0/50 |
| | the threshold | | |

^{a}cTnT of 35 ng/L can be extrapolated to hs-TnT of 50 ng/L.11
^{b}cTnT (reported in the European Modification of the Mayo Staging system as ng/mL) has been converted to ng/L to maintain consistency with units for hs-TnT, which may be used as a prognostic biomarker of cardiac response.
cTnT, cardiac troponin T; hs-TnT, high-sensitivity troponin T; NT-proBNP, N-terminal pro-brain natriuretic peptide.

These international, multicenter, double-blind, randomized, phase 3 trials were initiated in 2020 and are enrolling patients at over 70 sites in 14 countries (Canada, United States, United Kingdom, Belgium, France, Spain, Italy, Greece, Germany, Poland, Israel, Russian Federation, Japan and Australia).

Screening took up to 28 days. Then, patients in Mayo Stages IIIb (N=111) and IIIa (N=267) were randomized 2:1 to receive once-weekly intravenous infusions of the antibody described herein at 1000 mg/m²) or placebo for 4 weeks, followed by maintenance dosing every two weeks. In Study 301 (Stage IIIb), there were 74 patients in the antibody+SoC anti-PCD treatment group. In the Study 302 (Stage IIIa), there were 178 patients in the antibody+SoC anti-PCD treatment group. In the Study 301 (Stage IIIb), there were 37 patients in the placebo+SoC anti-PCD treatment group. In the Study 302 (Stage IIIa), there were 89 patients in the placebo+SoC anti-PCD treatment group. Patients received concurrent anti-PCD therapy according to the intuitional protocol for SoC. Treatment planned for a minimum duration of ≥50 weeks (12 months) or until the patient's death. As these are event-driven studies, treatment was continued to a minimum of 54 deaths for Study 301 and 77 deaths for Study 302. Patients were followed until death from any cause or until the end of the study.

The primary endpoint was overall survival, defined as the time from randomization to date of death, with censoring at last known living date (overall survival will be analyzed using time-to-event log-rank statistic). Key secondary endpoints were changes from baseline to week 50 (12 months/1 year) on the following tasks: functional status as measured by 6MWT (6-minute Walk Test), cardiac function as measured by GLS % (global longitudinal strain), quality of life as measured by KCCQ-OS (Kansas City Cardiomyopathy Questionnaire, overall score), quality of life as measured by SF-36v2 (Short-Form 36 version 2), and various safety indicia. Safety indicia included TEAE (treatment-emergent adverse event), clinical laboratory tests [changes in NT-proBNP, cTnT, free light chain (FLC), pharmacokinetics (PK)], immunogenicity laboratory tests, physical examination and vital signs, and 12-lead electrocardiogram. A subset of patients underwent contrast MRI (magnetic resonance imaging) of the heart with additional visualization of the liver and spleen at selected centers. Starting at Week 14, patients were assessed for changes in safety measurements, 6MWT, and QoL questionnaire every 12 weeks. Patients underwent echocardiography for global longitudinal strain (GLS) measurements and collection and analysis of 24-hour urine for protein assessment. Patient demographics at baseline are given in Table 19. Disease characteristics at baseline are given in Table 20.

TABLE 19

| | Study 301: | Study 302: |
|---|---|---|
| | Mayo Stage IIIb | Mayo Stage IIIa |
| Parameter | n = 19 | n = 58 |
| Age | n = 16 | n = 55 |
| Mean (SD), y | 66.6 (10.23) | 60.8 (16.91) |
| Sex, n (%) | | |
| Male | 9 (47.4) | 38 (65.5) |
| Female | 8 (42.1) | 18 (31.0) |
| Missing | 2 (10.5) | 2 (3.4) |
| Race,^{a} n (%) | | |
| Black or African American | 1 (5.3) | 5 (8.6) |
| White or Caucasian | 11 (57.9) | 40 (69.0) |
| Asian | 2 (10.5) | 8 (13.8) |
| Other | 2 (10.5) | 2 (3.4) |
| Missing | 3 (15.8) | 3 (5.2) |
| Ethnicity,^{a} n (%) | | |
| Hispanic or Latino | 1 (5.3) | 1 (1.7) |
| Not Hispanic or Latino | 14 (73.7) | 49 (84.5) |
| Other | 4 (21.1) | 8 (13.8) |

^{a}Patient self-reported
SD, standard deviation

TABLE 20

| | Study 301: | Study 302: |
|---|---|---|
| | Mayo Stage IIIb | Mayo Stage IIIa |
| Parameter | n = 19 | n = 58 |
| NT-proBNP | n = 14 | n = 39 |
| Median (range), pg/mL | 12,956 (7008- | 5049 (1086- |
| | 32509) | 28052) |
| hs-TnT | n = 14 | n = 40 |
| Median (range), ng/L | 101.6 (49.15- | 109.8 (19.41- |
| | 280.4) | 1164.0) |
| eGFR | n = 6 | n = 22 |
| Median (range), mL/min/1.73 m² | 40 (29-89) | 67.5 (33-105) |
| Proteinuria | n = 10 | n = 29 |
| Median (range), g/24 h | 0.159 (0.032- | 0.252 (0.0001- |
| | 14.879) | 12.240) |

eGFR, estimated glomerular filtration rate; hs-TnT, high-sensitivity troponin T; NT-proBNP, N-terminal pro-brain natriuretic peptide.

High levels of cardiac biomarkers and poor eGFR indicate extensive cardiac and/or renal involvement.

NT-proBNP levels were >332 μg/mL threshold for patients in Mayo Stage Ma (Study 302) and >8500 μg/mL for patients in Mayo Stage IIIb (Study 301). At least 14/19 (74%) patients in Study 301 and 47/58 (81%) patients in Study 302 have received at least 4 doses of the antibody described herein concurrently with anti-PCD therapy.

These ongoing trials evaluate the efficacy and safety of the antibody described herein as first-in-class treatment to reduce amyloid burden in patients with cardiac AL amyloidosis. A subset of patients who consent to participation in the cardiac magnetic resonance imaging (MRI) substudy undergo contrast MRI of the heart with additional visualization of the liver and spleen at selected center. Appropriate disease management and optimal treatment outcomes can be achieved only if accurate prognostic information is available. Patient classification in the 301 and 302 studies is based on the 2013 European Modification of Mayo 2004 Staging system, which is robust and has greater accuracy compared with other staging systems in distinguishing between patients with less severe disease and critically ill patients with extensive cardiac involvement and a greater risk of mortality. Notably, the 301 study in Mayo Stage IIIb is the first randomized, placebo-controlled, efficacy clinical trial to formally assess the effects of a pharmacological agent in this severely ill population.

Although the disclosure has been described with reference to the above examples, it will be understood that modifications and variations are encompassed within the spirit and scope of the disclosure. Accordingly, the disclosure is limited only by the following claims.

1. A method of treating multiple myeloma in a subject comprising:
administering to the subject
(a) an antibody having a heavy chain variable domain (VH) as set forth in SEQ ID NO: 1 and a light chain variable domain (VL) as set forth in SEQ ID NO: 2; and
(b) an anti-CD38 antibody;
thereby treating multiple myeloma in the subject.

2. The method of claim 1, wherein the antibody is administered in a dose from about 250 mg/m² to 1,375 mg/m².

3. The method of claim 2, wherein the antibody dose is selected from about 500 mg/m², about 750 mg/m², about 1,000 mg/m².

4. The method of claim 1, wherein a weekly antibody administration dose comprises about 10 to 15 mg/kg of antibody, about 15 to 20 mg/kg of antibody or about 20 to 30 mg/kg of antibody.

5. The method of claim 1, wherein administering about 500 mg/m² of antibody comprises administering about 1,000 to 1,500 mg of antibody, administering about 750 mg/m² of antibody comprises administering about 1,500 to 2,500 mg of antibody, and administering about 1,000 mg/m² of antibody comprises administering about 2,500 to 3,000 mg of antibody.

6. The method of claim 1, wherein the 500 mg/m², 750 mg/m² and 1,000 mg/m² antibody administration dose achieves a site occupancy of a target receptor of at least 90%.

7. The method of claim 1, wherein the antibody is administered weekly for at least 2, 3 or 4 weeks.

8. The method of claim 1, further comprising administering a maintenance dose of the antibody to the subject thereafter.

9. The method of claim 8, wherein the maintenance dose of the antibody is administered biweekly, triweekly, or monthly after the first 2, 3, 4 or more weeks.

10. The method of claim 1, wherein the anti-CD38 antibody is selected from Daratumumab, Isatuximab, CID-103 (CASI Pharma), or Moro3087 (Morphosys) or a combination thereof.

11. The method of claim 10, wherein the daratumumab is administered in a dose from about 10 to 20 mg/kg.

12. The method of claim 10, wherein the daratumumab is administered weekly for at least a first cycle or second cycle.

13. The method of claim 12, further comprising administering a maintenance dose of the daratumumab to the subject thereafter.

14. The method of claim 13, wherein the maintenance dose of the daratumumab is administered biweekly, triweekly, every four weeks or monthly after the first cycle or second cycle.

15. The method of claim 10, wherein the antibody is administered prior to, simultaneously with, or after administration of the daratumumab.

16. The method of claim 10, wherein the antibody is administered prior to the administration of the daratumumab.

17. The method of claim 10, wherein the antibody and/or the daratumumab is administered by intravenous (IV) infusion, subcutaneous injection or intramuscular injection.

18. The method of claim 1, wherein administering the antibody induces removal of amyloid deposits present in an organ or tissue.

19. The method of claim 18, wherein the organ or tissue is selected from the group consisting of heart, kidney, liver, lung, gastrointestinal tract, nervous system, muscular skeletal system, soft tissue, skin and any combination thereof.

20. (canceled)

21. The method of claim 1, wherein the antibody is administered to the subject in a pharmaceutical composition further comprising:
(a) one or more isotonic agents; and
(b) a non-ionic surfactant.

22. The method of claim 21, wherein the pharmaceutical composition comprises from about 20 to 40 mg/mL antibody.

23. The method of claim 21, wherein the isotonic agent is sodium acetate; the buffer is sodium chloride; the non-ionic surfactant is polysorbate 80.

24. The method of claim 21, wherein the pharmaceutical composition comprises from about 15 to 35 mM sodium acetate.

25. The method of claim 21, wherein the pharmaceutical composition comprises from about 25 to 75 mM sodium chloride.

26. The method of claim 21, wherein the pharmaceutical composition comprises from about 0.5 to 5% mannitol.

27. The method of claim 21, wherein the pharmaceutical composition comprises from about 0.001 to 0.1% polysorbate 80.

28. The method of claim 21, wherein the pharmaceutical composition has a pH from about 5 to 6.

29. (canceled)

30. The method of claim 1, wherein the antibody is a mixture comprising a native fraction, a reduced fraction, and/or a glycosylated or deglycosylated fraction, each having a heterogeneous charge.

31. (canceled)

32. The method of claim 30, wherein the reduced fraction comprises light chains with glycated lysines.

33. The method of claim 1, wherein the antibody is a mixture comprising intact antibodies, halfmer fragments, incomplete antibody fragments, other fragments and/or aggregates thereof.

34-36. (canceled)

37. A method of inhibiting amyloid formation by binding to precursor misfolded proteins in the circulation of a subject comprising:
administering to the subject
(a) an antibody having a heavy chain variable domain (VH) as set forth in SEQ ID NO:1 and a light chain variable domain (VL) as set forth in SEQ ID NO:2; and
(b) an anti-CD38 antibody selected from Daratumumab, Isatuximab, CID-103 (CASI Pharma), or Moro3087 (Morphosys) or a combination thereof, thereby inhibiting amyloid formation in the subject.

38. The method of claim 37, wherein the antibody is administered in a dose from about 250 mg/m² to 1,375 mg/m².

39. The method of claim 37, wherein the antibody dose is selected from about 500 mg/m², about 750 mg/m², and about 1,000 mg/m².

40. The method of claim 37, wherein a weekly antibody administration dose of about 500 mg/m² of antibody comprises about 10 to 15 mg/kg of antibody, a weekly dose of about 750 mg/m² comprises about 15 to 20 mg/kg of antibody and a weekly dose of about 1,000 mg/m² of antibody comprises about 20 to 30 mg/kg of antibody.

41-72. (canceled)

73. The method of claim 1, wherein the antibody is administered in a dose from about 6 mg/kg to 32 mg/kg.

74-75. (canceled)

76. The method of claim 37, wherein the antibody is administered in a dose from about 6 mg/kg to 32 mg/kg.

77-78. (canceled)


SEQUENCE LISTING (10 sequences)

SEQ ID NO: 1 - protein, 111 aa, synthetic construct
  source 1..111: note = Monoclonal antibody variable sequences - VH region
QVQLKESGPGLVAPSQSLSITCTVSGFSLSSYGVSWVRQPPGKGLEWLGVIWGDGSTNYHPNLMSRLSISKDISKSQVLFKLNSLQTDDTATYYCVTLDYWGQGTSVTVSS

SEQ ID NO: 2 - protein, 112 aa, synthetic construct
  source 1..112: note = Monoclonal antibody variable sequences - VK region
DVVMTQTPLSLPVSLGDQASISCRSSQSLVHRNGNTYLHWYLQKPGQSPKLLIYKVSNRFSGVPDRFSGSGSGTDFTLKISRVEAEDLGLYFCFQTTYVPNTFGGGTKLEIK

SEQ ID NO: 3 - protein, 17 aa, synthetic construct
  source 1..17: note = Monoclonal antibody CDR sequences CDRL1
SSQSLVHRNGNTYLHWY

SEQ ID NO: 4 - protein, 6 aa, synthetic construct
  source 1..6: note = Monoclonal antibody CDR sequences CDRL2
KVSNRF

SEQ ID NO: 5 - protein, 6 aa, synthetic construct
  source 1..6: note = Monoclonal antibody CDR sequences CDRL3
QTTYVP

SEQ ID NO: 6 - protein, 7 aa, synthetic construct
  source 1..7: note = Monoclonal antibody CDR sequences CDRH1
SYGVSWV

SEQ ID NO: 7 - protein, 12 aa, synthetic construct
  source 1..12: note = Monoclonal antibody CDR sequences CDRH2
PNLMSRLSISKD

SEQ ID NO: 8 - protein, 6 aa, synthetic construct
  source 1..6: note = Monoclonal antibody CDR sequences CDRH3
DYWGQG

SEQ ID NO: 9 - protein, 441 aa, synthetic construct
  source 1..441: note = Protein Sequence of Heavy Chain
QVQLKESGPGLVAPSQSLSITCTVSGFSLSSYGVSWVRQPPGKGLEWLGVIWGDGSTNYHPNLMSRLSISKDISKSQVLFKLNSLQTDDTATYYCVTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, synthetic construct
  source 1..219: note = Protein Sequence of Light Chain
DVVMTQTPLSLPVSLGDQASISCRSSQSLVHRNGNTYLHWYLQKPGQSPKLLIYKVSNRFSGVPDRFSGSGSGTDFTLKISRVEAEDLGLYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC